(19) United States
(12) Patent Application Publication
(10) Pub. No.: US 2024/0269099 A1
(43) Pub. Date: Aug. 15, 2024

(54) METHODS AND COMPOSITIONS FOR TREATING CANCER

(71) Applicant: Onconova Therapeutics, Inc., Newtown, PA (US)

(72) Inventors: Steven Fruchtman, New York, NY (US); Matthew Parris, Plainsboro, NJ (US)

(21) Appl. No.: 18/542,101

(22) Filed: Dec. 15, 2023

Related U.S. Application Data
(63) Continuation of application No. PCT/US22/33657, filed on Jun. 15, 2022.
(60) Provisional application No. 63/211,070, filed on Jun. 16, 2021.
(60) Provisional application No. 63/211,071, filed on Jun. 16, 2021.

(51) Int. Cl. A61K 31/197; A61K 45/06; A61P 35/00

(52) U.S. Cl. CPC A61K 31/197; A61K 45/06; A61P 35/00

(57) ABSTRACT

RAS proteins are frequently mutated in human cancers. Disclosed herein are compounds that disrupt RAS effectors and inhibits Ras/Raf/MEK/ERK pathway signaling. Further disclosed herein are methods of using compounds in combination with checkpoint inhibitors to treat cancer.

### CROSS-REFERENCE

This application is a continuation of International Application PCT/US22/33657, filed Jun. 15, 2022, which claims the benefit of U.S. Provisional Application No. 63/211,070 filed Jun. 16, 2021, and U.S. Provisional Application No. 63/211,071 filed Jun. 16, 2021, each of which is incorporated herein by reference in their entirety.

### BACKGROUND

A large subset of lung adenocarcinomas has a KRAS mutation as the predominant genetic driver. Tumors carrying a mutation in KRAS can have a worse prognosis than KRAS wild-type tumors. Thus, therapies that can target the KRAS pathway could be beneficial for patients with cancers having KRAS mutations.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

Disclosed herein is a method of treating a condition comprising administering to a subject in need thereof: a) a therapeutically-effective amount of a compound of the formula:

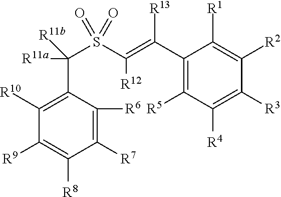

or a pharmaceutically-acceptable salt or zwitterion thereof,
   wherein:
each R¹, R², R³, R⁴, R⁵, R⁶, R⁷, R⁸, R⁹, R¹⁰, R^{11a}, R^{11b}, R¹², and R¹³ is independently alkyl, alkenyl, alkynyl, alkoxy, aryl, heteroaryl, heterocyclyl, —C(O)R^{x}, —C(O)OR^{x}, —C(O)NR^{x}R^{y}, —OR^{x}, —SR^{x}, —NR^{x}R^{y}, —NR^{x}C(O)R^{y}, —OC(O)R^{x}, or —SiR^{x}R^{y}R^{z}, each of which is independently substituted or unsubstituted; or hydrogen or halogen; and
each R^{x}, R^{y}, and R^{z} is independently alkyl, alkenyl, alkynyl, aryl, heteroaryl, or heterocyclyl, each of which is independently substituted or unsubstituted; or hydrogen or halogen, and
   b) a therapeutically-effective amount of a checkpoint inhibitor.

### BRIEF DESCRIPTION OF THE DRAWINGS

FIG. 1 is a schematic of a dose escalation study of a compound disclosed herein.

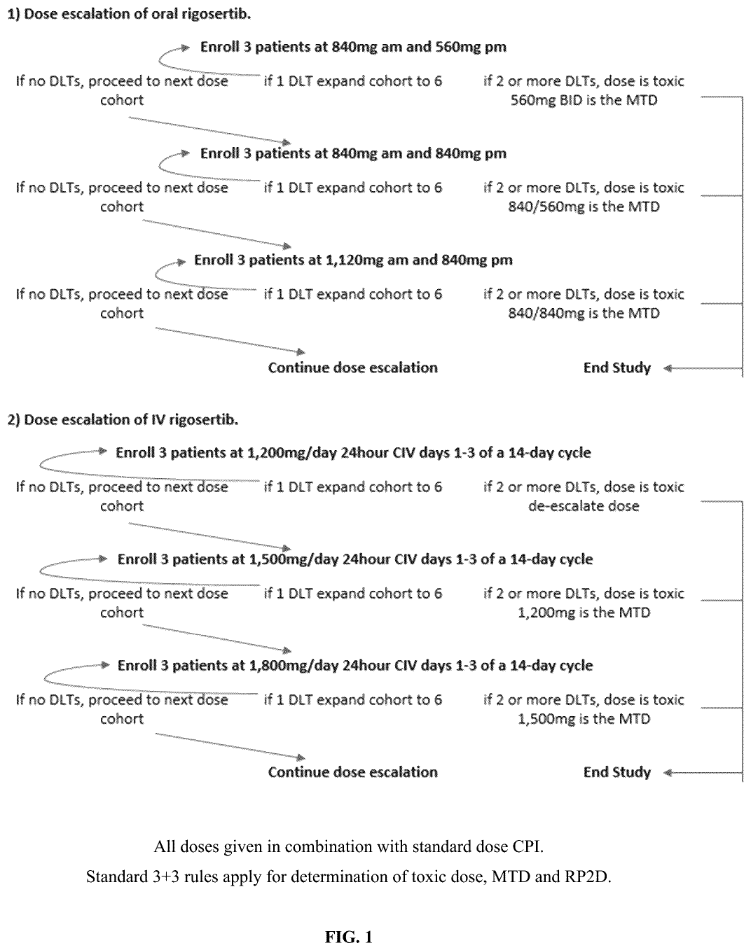

### DETAILED DESCRIPTION OF THE INVENTION

Ras is the most frequently mutated gene causing cancer. Approximately 40% of patients with non-small cell lung cancer (NSCLC) have a mutation of RAS, most frequently KRAS. A mechanism of action for a compound disclosed herein, for example, rigosertib, is to down-modulate the mutated RAS pathway. This modulation of the RAS pathway can result in less production of ERK, a protein involved in cell proliferation. When ERK is overexpressed. ERK can provide a proliferative advantage to cells, thus leading to cancer. KRAS-mutated NSCLC can be managed with, for example, a checkpoint inhibitor.

Rigosertib ((E)-2-(5-((2,4,6-trimethoxystyrylsulfonyl)methyl)-2-methoxy phenylamino)acetic acid, or a pharmaceutically-acceptable salt or zwitterion thereof) can block the RAS cascade and promote the expression of novel antigens on the tumor's surface. This process can turn cold tumors that are not surrounded by host lymphocytes into hot tumors that are surrounded by host lymphocytes. This modulation of the tumor micro-environment (TME) in combination with the checkpoint blockade can facilitate the host immune system to contribute to tumor control. The host lymphocytes can contribute to tumor control when exposed to a checkpoint inhibitor.

The present disclosure provides a combination of a compound disclosed herein, for example, rigosertib, with a checkpoint inhibitor. A checkpoint inhibitor disclosed herein can be, for example, nivolumab, which blocks PD-1.

In some embodiments, rigosertib can cause genitourinary toxicity including dysuria and hematuria. In some embodiments, risk mitigation strategies can minimize these effects.

In some embodiments, the dose of the combination partner of the checkpoint inhibitor is the dose as per the FDA label. In some embodiments, patient safety requires a deviation from the label dose.

### Compounds of the Disclosure

In some embodiments, disclosed herein is a compound of the formula:

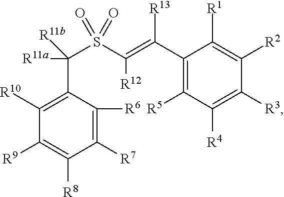

wherein:
each R¹, R², R³, R⁴, R⁵, R⁶, R⁷, R⁸, R⁹, R¹⁰, R^{11a}, R^{11b}, R¹², and R¹³ is independently alkyl, alkenyl, alkynyl, alkoxy, aryl, heteroaryl, heterocyclyl, —C(O)R^{x}, —C(O)OR^{x}, —C(O)NR^{x}R^{y}, —OR^{x}, —SR^{x}, —NR^{x}R^{y}, —NR^{x}C(O)R^{y}, —OC(O)R^{x}, or —SiR^{x}R^{y}R^{z}, each of which is independently substituted or unsubstituted; or hydrogen or halogen; and
each R^{x}, R^{y}, and R^{z} is independently alkyl, alkenyl, alkynyl, aryl, heteroaryl, or heterocyclyl, each of which is independently substituted or unsubstituted; or hydrogen or halogen,
   or a pharmaceutically-acceptable salt or zwitterion thereof.

In some embodiments, R¹, R³, and R⁵ are the same. In some embodiments, R¹, R³, R⁵, and R⁸ are the same. In some embodiments, each R¹, R³, and R⁵ is independently OR^{x}. In some embodiments, each R¹, R³, R⁵ and R⁸ is independently OR^{x}. In some embodiments, each R^{x} is independently alkyl, aryl, heteroaryl, heterocyclyl, each of which is independently substituted or unsubstituted. In some embodiments, each R^{x} is independently hydrogen. In some embodiments, each R is independently unsubstituted C₁₋₆ alkyl. In some embodiments, each R^{x} is independently unsubstituted C₁₋₃ alkyl. In some embodiments, each R^{x} is independently methyl. In some embodiments, each R^{x} is independently ethyl. In some embodiments, each R^{x} is independently substituted C₁₋₆ alkyl. In some embodiments, each R^{x} is independently substituted C₁₋₃ alkyl. In some embodiments, each R^{x} is C₁ alkyl substituted with hydroxy, sulfhydryl, halogen, an amino group, a nitro group, cyano, a sulfoxide group, a sulfone group, a sulfonamide group, a carboxyl group, a carboxylic acid, a carboxaldehyde group, alkoxy, aryl, heterocyclyl groups, acyl groups, amide, or an ester.

In some embodiments, R² is hydrogen. In some embodiments, R⁴ is hydrogen. In some embodiments, R⁶ is hydrogen. In some embodiments, R⁹ is hydrogen. In some embodiments, R¹⁰ is hydrogen.

In some embodiments, R⁷ is alkyl, alkoxy, aryl, heteroaryl, heterocyclyl, OR^{x}, or NR^{x}R^{y}. In some embodiments, R⁷ is NR^{x}R^{y}. In some embodiments, R^{x} is hydrogen. In some embodiments, R^{y} is hydrogen. In some embodiments, R^{y} is substituted alkyl. In some embodiments, R^{y} is substituted C₁₋₆ alkyl. In some embodiments, R^{y} is C₁ alkyl substituted with hydroxy, sulfhydryl, halogen, an amino group, a nitro group, cyano, a sulfoxide group, a sulfone group, a sulfonamide group, a carboxyl group, a carboxylic acid, a carboxaldehyde group, alkoxy, aryl, a heterocyclyl group, an acyl group, amide, or an ester. In some embodiments, R^{y} is CH₂COOH.

In some embodiments, each R^{11a} and R^{11b} is independently alkyl, aryl, heteroaryl, or heterocyclyl, each of which is independently substituted or unsubstituted. In some embodiments, each R^{11a} and R^{11b} is independently substituted C₁₋₆ alkyl. In some embodiments, each R^{11a} and R^{11b} is independently unsubstituted C₁₋₆ alkyl. In some embodiments, each R¹² and R¹³ is independently alkyl, aryl, heteroaryl, heterocyclyl, each of which is independently substituted or unsubstituted. In some embodiments, each R¹² and R¹³ is independently substituted C₁₋₆ alkyl. In some embodiments, each R¹² and R¹³ is independently unsubstituted C₁₋₆ alkyl. In some embodiments, R^{11a} is hydrogen. In some embodiments, R^{11b} is hydrogen. In some embodiments, R¹² is hydrogen. In some embodiments, R¹³ is hydrogen.

In some embodiments, disclosed herein is a compound of the formula:

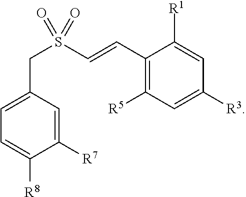

In some embodiments, the compound has the formula:

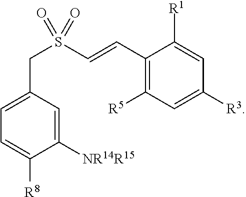

In some embodiments, each R¹, R³, R⁵, and R⁸ is independently OR^{x}. In some embodiments, each R^{y} is independently alkyl, aryl, heteroaryl, heterocyclyl, each of which is independently substituted or unsubstituted. In some embodiments, each R^{x} is independently hydrogen. In some embodiments, each R is independently unsubstituted C₁₋₆ alkyl. In some embodiments, each R is independently unsubstituted C₁₋₃ alkyl. In some embodiments, each R^{x} is independently methyl. In some embodiments, each R^{x} is independently ethyl. In some embodiments, each R^{x} is independently substituted C₁₋₆ alkyl. In some embodiments, each R^{x} is independently substituted C₁₋₃ alkyl. In some embodiments, each R^{x} is independently methyl that is substituted.

In some embodiments, each R¹⁴ and R¹⁵ is independently alkyl, alkoxy, aryl, heteroaryl, heterocyclyl, each of which is independently substituted or unsubstituted; or hydrogen. In some embodiments, R¹⁴ is H. In some embodiments, R¹⁵ is H. In some embodiments, R¹⁵ is substituted alkyl. In some embodiments, R¹⁵ is substituted C₁₋₆ alkyl. In some embodiments, R¹⁵ is substituted C₁ alkyl. In some embodiments, R¹⁵ is CH₂COOH.

In some embodiments, the compound has the formula:

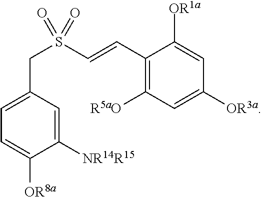

In some embodiments, each R^{1a}, R^{3a}, R^{5a}, and R^{8a} is the same. In some embodiments, each R^{1a}, R^{3a}, R^{5a}, and R^{8a} is different. In some embodiments, each R^{1a}, R^{3a}, R^{5a}, and R^{8a} is independently substituted or unsubstituted alkyl. In some embodiments, each R^{1a}, R^{3a}, R^{5a}, and R^{8a} is independently substituted or unsubstituted C₁₋₈ alkyl. In some embodiments, each R^{1a}, R^{3a}, R^{5a}, and R^{8a} is independently substituted or unsubstituted C₁₋₃ alkyl. In some embodiments, each R^{1a}, R^{3a}, R^{5a}, and R^{8a} is independently substituted or unsubstituted C₁ alkyl. In some embodiments, each R^{1a}, R^{3a}, R^{5a}, and R^{8a} is independently methyl. In some embodiments, each R^{1a}, R^{3a}, R^{5a}, and R^{8a} is independently ethyl.

In some embodiments, each R¹⁴ and R¹⁵ is independently alkyl, alkoxy, aryl, heteroaryl, heterocyclyl, each of which is independently substituted or unsubstituted; or hydrogen. In some embodiments, R¹⁴ is H. In some embodiments, R¹⁵ is H. In some embodiments, R¹⁵ is substituted alkyl. In some embodiments, R¹⁵ is substituted C₁₋₆ alkyl. In some embodiments, R¹⁵ is substituted C₁ alkyl. In some embodiments, R¹⁵ is CH₂COOH.

In some embodiments, the compound has the formula:

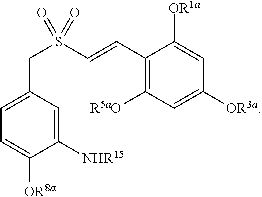

In some embodiments, each R^{1a}, R^{3a}, R^{5a}, and R^{8a} is independently substituted or unsubstituted alkyl. In some embodiments, each R^{1a}, R^{3a}, R^{5a}, and R^{8a} is independently substituted or unsubstituted C₁₋₈ alkyl. In some embodiments, R^{1a} is C₁₋₆ alkyl. In some embodiments, R^{3a} is C₁₋₆ alkyl. In some embodiments, R^{5a} is C₁₋₆ alkyl. In some embodiments, R^{8a} is C₁₋₆ alkyl. In some embodiments, R^{1a} is methyl. In some embodiments, R^{3a} is methyl. In some embodiments, R^{5a} is methyl. In some embodiments, R^{8a} is methyl.

In some embodiments, R¹⁵ is substituted alkyl. In some embodiments, R¹⁵ is substituted C₁₋₆ alkyl. In some embodiments, R¹⁵ is substituted C₁ alkyl. In some embodiments, R¹⁵ is CH₂COOH.

In some embodiments, disclosed herein is a compound of the formula:

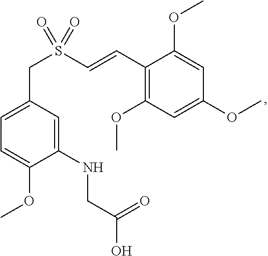 [SMILES: COc1cc(OC)c(/C=C/S(=O)(=O)Cc2ccc(OC)c(NCC(=O)O)c2)c(OC)c1]

(E)-2-(5-((2,4,6-trimethoxystyrylsulfonyl)methyl)-2-methoxyphenylamino)acetic acid, or a pharmaceutically-acceptable salt or zwitterion thereof. In some embodiments, the compound is

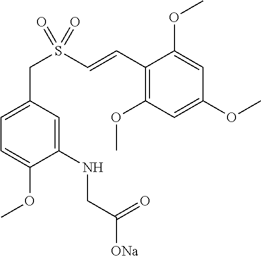

sodium (E)-2-(5-((2,4,6-trimethoxystyrylsulfonyl)methyl)-2-methoxyphenylamino)acetate. In some embodiments, a compound disclosed herein is a sodium salt.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

Non-limiting examples of alkyl and alkylene groups include straight, branched, and cyclic alkyl and alkylene groups. An alkyl or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. In some embodiments, alkyl or alkylene is C₁₋₈ alkyl or C₁₋₈ alkylene that is substituted or unsubstituted. In some embodiments, alkyl or alkylene is C₁₋₆ alkyl or C₁₋₆ alkylene that is substituted or unsubstituted. In some embodiments, alkyl or alkylene is C₁₋₃ alkyl or C₁₋₃ alkylene that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxy cyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxy buten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxy phenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, and 4-(N,N-diethylamino)phenyl.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl. [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

In some embodiments, the compound is at least about 85% pure. In some embodiments, the compound is at least about 90% pure. In some embodiments, the compound is at least about 95% pure. In some embodiments, the compound is at least about 98% pure. In some embodiments, the compound is at least about 99% pure. In some embodiments, the compound is at least about 99.5% pure.

### Pharmaceutically Acceptable Salts

The method disclosed herein provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is a sodium salt.

Metal salts can arise from the addition of an inorganic base to a compound disclosed herein. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Acid addition salts can arise from the addition of an acid to a compound disclosed herein. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

### Pharmaceutical Compositions of the Disclosure

A pharmaceutical composition of a compound disclosed herein can be a combination of any pharmaceutical compounds described herein with other chemical components, such as carriers, stabilizers, diluents, dispersing agents, suspending agents, thickening agents, or excipients. The pharmaceutical composition facilitates administration of the compound to an organism. Pharmaceutical compositions can be administered in therapeutically-effective amounts as pharmaceutical compositions by various forms and routes including, for example, intravenous, intravitreal, intranasal, inhalation, nasal inhalation, mouth inhalation, intratracheal, intrapulmonary, transmucosal, subcutaneous, intramuscular, oral, rectal, aerosol, parenteral, ophthalmic, pulmonary, transdermal, vaginal, otic, nasal, and topical administration.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated readily by combining the active compounds with pharmaceutically-acceptable carriers or excipients. Such carriers can be used to formulate tablets, powders, pills, dragees, capsules, liquids, gels, syrups, elixirs, slurries, or suspensions for oral ingestion by a subject.

Pharmaceutical preparations for oral use can be obtained by mixing one or more solid excipient with one or more compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used, which can contain an excipient such as gum arabic, talc, polyvinylpyrrolidone, carbopol gel, polyethylene glycol, or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline-processed. The push-fit capsules can contain the active ingredients in admixture with filler such as lactose, binders such as starches, or lubricants such as talc or magnesium stearate, and stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

An active compound can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers, and preservatives.

Formulations suitable for transdermal administration of the active compounds can employ transdermal delivery devices and transdermal delivery patches, and can be lipophilic emulsions or buffered aqueous solutions, dissolved or dispersed in a polymer or an adhesive. Such patches can be constructed for continuous, pulsatile, or on demand delivery of pharmaceutical compounds. Transdermal delivery can be accomplished by iontophoretic patches. Additionally, transdermal patches can provide controlled delivery. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically-acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin or the eye.

For administration by inhalation, the active compounds can be in a form as an aerosol, a vapor, a mist, or a powder. Inhalation can occur through by nasal delivery, oral delivery, or both. Nasal or intranasal administration involves insufflation of compounds through the nose, for example, nasal drops and nasal sprays. This route of administration can result in local and/or systemic effects. Inhaler or insufflator devices can be used for nose-to-lung delivery of compounds described herein.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, and synthetic polymers such as polyvinylpyrrolidone and PEG. In suppository forms of the compositions, a low-melting point wax such as a mixture of fatty acid glycerides or cocoa butter, can be used. In some embodiments, a pharmaceutical composition of the disclosure comprises PEG. In some embodiments, a pharmaceutical composition of the disclosure comprises PEG-400. In some embodiments, a pharmaceutical composition of the disclosure comprises PEG-4000.

In practicing a method of treatment or use provided herein, therapeutically-effective amounts of a compound described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compound described herein can be manufactured, for example, by mixing, dissolving, granulating, dragee-making, levigating, emulsifying, encapsulating, entrapping, or compression processes.

The pharmaceutical compositions can include at least one pharmaceutically-acceptable carrier, diluent, or excipient and compound described herein as free-base or pharmaceutically-acceptable salt form. The methods and pharmaceutical compositions described herein include the use of crystalline forms (also known as polymorphs), and active metabolites of these compounds having the same type of activity.

Methods for the preparation of compositions comprising a compound described herein include formulating a compound with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in a method disclosed herein include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Non-limiting examples of pharmaceutically-acceptable excipients suitable for use in the method disclosed herein include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, anti-microbial agents, plant cellulosic material and spheronization agents, and any combination thereof.

A composition of a compound disclosed herein can be, for example, an immediate release form or a controlled release formulation. An immediate release formulation can be formulated to allow a compound to act rapidly. Non-limiting examples of immediate release formulations include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

The disclosed compositions can optionally comprise from about 0.001% to about 0.005% weight by volume pharmaceutically-acceptable preservatives.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16, or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), each of which is incorporated by reference in its entirety.

A method disclosed herein includes, for example, administration of a compound disclosed herein, or a pharmaceutically-acceptable salt thereof, in combination with a pharmaceutically-acceptable carrier. The carrier can be selected to minimize any degradation of the active ingredient and to minimize any adverse side effects in the subject.

A compound disclosed herein or a pharmaceutically-acceptable salt thereof disclosed herein can be conveniently formulated into pharmaceutical compositions composed of one or more pharmaceutically-acceptable carriers. See e.g., Remington's Pharmaceutical Sciences, latest edition, by E.W. Martin Mack Pub. Co., Easton, PA, which discloses carriers and methods of preparing pharmaceutical compositions that can be used in conjunction with the preparation of formulations of the compound described herein and which is incorporated by reference herein. Such pharmaceuticals can be standard carriers for administration of compositions to humans and non-humans, including solutions such as sterile water, saline, and buffered solutions at physiological pH. Other compositions can be administered according to standard procedures. For example, pharmaceutical compositions can also include one or more additional active ingredients such as antimicrobial agents, anti-inflammatory agents, and anesthetics.

Non-limiting examples of pharmaceutically-acceptable carriers include saline solution, Ringer's solution and dextrose solution. Further carriers include sustained release preparations such as semipermeable matrices of solid hydrophobic polymers containing the compound disclosed herein or a pharmaceutically-acceptable salt thereof, where the matrices are in the form of shaped articles, such as films, liposomes, microparticles, and microcapsules.

A method disclosed herein relates to administering the compound disclosed herein or a pharmaceutically-acceptable salt thereof as part of a pharmaceutical composition. In various embodiments, compositions of a compound disclosed herein can comprise a liquid comprising an active agent in solution, in suspension, or both. Liquid compositions can include gels. In one embodiment, the liquid composition is aqueous. Alternatively, the composition can take form of an ointment. In another embodiment, the composition is an in situ gellable aqueous composition. In some embodiments, the composition is an in situ gellable aqueous solution.

Pharmaceutical formulations can include additional carriers, as well as thickeners, diluents, buffers, preservatives, and surface active agents in addition to a compound disclosed herein. Pharmaceutical formulations can also include one or more additional active ingredients such as antimicrobial agents, anti-inflammatory agents, and anesthetics.

An excipient can fill a role as simple and direct as being an inert filler, or an excipient as used herein can be part of a pH stabilizing system or coating to insure delivery of the ingredients safely to the stomach.

The compound disclosed herein or a pharmaceutically-acceptable salt thereof can also be present in liquids, emulsions, or suspensions for delivery of active therapeutic agents in aerosol form to cavities of the body such as the nose, throat, or bronchial passages.

Depending on the intended mode of administration, the pharmaceutical compositions administered as part of a method disclosed herein can be in the form of solid, semi-solid or liquid dosage forms, such as, for example, tablets, suppositories, pills, capsules, powders, liquids, suspensions, lotions, creams, gels, for example, in unit dosage form suitable for single administration of a precise dosage. The compositions can contain, as noted above, an effective amount of the compound disclosed herein or a pharmaceutically-acceptable salt thereof in combination with a pharmaceutically-acceptable carrier and, in addition, can include other medicinal agents, pharmaceutical agents, carriers, adjuvants, diluents, etc.

In some embodiments, a pharmaceutical composition can have a pH of from about 7 to about 12, from about 9 to about 13, from about 3 to about 4, from about 4 to about 5, from about 5 to about 6, from about 6 to about 7, from about 7 to about 8, from about 8 to about 9, from about 9 to about 10, from about 10 to about 11, from about 11 to about 12, from about 12 to about 13, or from about 13 to about 14. In some embodiments, a pharmaceutical composition can have a pH of at least about 4, at least about 5, at least about 6, at least about 7, at least about 8, at least about 9, at least about 10, at least about 11, at least about 12, or at least about 13. In some embodiments, a pharmaceutical composition can have a pH of about 4, about 5, about 6, about 7, about 8, about 9, about 10, about 11, about 12, or about 13. In some embodiments, a pharmaceutical composition can have a pH of at most about 4, at most about 5, at most about 6, at most about 7, at most about 8, at most about 9, at most about 10, at most about 11, at most about 12, or at most about 13.

A compound described herein can be present in a composition in a range of from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, or from about 250 mg to about 300 mg.

A compound described herein can be present in a composition in an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, or about 300 mg.

In some embodiments, a pharmaceutical composition of the disclosure can comprise a compound in an amount of from about 25 mg/mL to about 3000 mg/mL. In some embodiments, a pharmaceutical composition of the disclosure can comprise a compound in an amount of from about 25 mg/mL to about 50 mg/mL, from about 50 mg/mL to about 100 mg/mL, from about 100 mg/mL to about 250 mg/mL, from about 250 mg/mL to about 500 mg/mL, from about 500 mg/mL to about 750 mg/mL, from about 750 mg/mL to about 1000 mg/mL, from about 1000 mg/mL to about 1500 mg/mL, from about 1500 mg/mL to about 2000 mg/mL, from about 2000 mg/mL to about 2500 mg/mL, or from about 2500 mg/mL to about 3000 mg/mL. In some embodiments, a pharmaceutical composition of the disclosure can comprise a compound in an amount of at least about 25 mg/mL, at least about 50 mg/mL, at least about 100 mg/mL, at least about 250 mg/mL, at least about 500 mg/mL, at least about 750 mg/mL, at least about 1000 mg/mL, at least about 1250 mg/mL, at least about 1500 mg/mL, at least about 1750 mg/mL, at least about 2000 mg/mL, at least about 2250 mg/mL, at least about 2500 mg/mL, at least about 2750 mg/mL, or at least about 3000 mg/mL. In some embodiments, a pharmaceutical composition of the disclosure can comprise a compound in an amount of about 25 mg/mL, about 50 mg/mL, about 100 mg/mL, about 250 mg/mL, about 500 mg/mL, about 750 mg/mL, about 1000 mg/mL, about 1250 mg/mL, about 1500 mg/mL, about 1750 mg/mL, about 2000 mg/mL, about 2250 mg/mL, about 2500 mg/mL, about 2750 mg/mL, or about 3000 mg/mL. In some embodiments, a pharmaceutical composition of the disclosure can comprise a compound in an amount of at most about 25 mg/mL, at most about 50 mg/mL, at most about 100 mg/mL, at most about 250 mg/mL, at most about 500 mg/mL, at most about 750 mg/mL, at most about 1000 mg/mL, at most about 1250 mg/mL, at most about 1500 mg/mL, at most about 1750 mg/mL, at most about 2000 mg/mL, at most about 2250 mg/mL, at most about 2500 mg/mL, at most about 2750 mg/mL, or at most about 3000 mg/mL.

In some embodiments, a pharmaceutical composition of the disclosure can comprise a compound in an amount of about 35 mg/mL. In some embodiments, a pharmaceutical composition of the disclosure can comprise a compound in an amount of about 70 mg/mL. In some embodiments, a pharmaceutical composition of the disclosure can comprise a compound in an amount of about 75 mg/mL. In some embodiments, a pharmaceutical composition of the disclosure can comprise a compound in an amount of about 140 mg/mL. In some embodiments, a pharmaceutical composition of the disclosure can comprise a compound in an amount of about 280 mg/mL.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

Intravenous (IV) Formulation: In some embodiments, a compound or pharmaceutical composition of the disclosure can be administered intravenously. In some embodiments, a formulation disclosed herein can be, for example, a parenteral formulation of a solution of the active pharmaceutical ingredient (API) at a concentration of about 75 mg/mL in a liquid vehicle of polyethylene glycol (PEG) 400 with pH 7-13 or pH 12-13 adjusted by use of sodium hydroxide (NaOH) solution. The solution can be a clear, colorless to pale yellow, sterile, preservative-free solution packaged in a clear glass vial sealed with a Teflon-coated rubber stopper. A vial disclosed herein can be a 30-mL clear glass vial containing, for example, 24 mL of the parenteral formulation. Dilution with IV saline can be required when the parenteral formulation is administered IV.

Oral Capsule Formulation: In some embodiments, a compound or pharmaceutical composition of the disclosure can be administered orally. In some embodiments, a compound or pharmaceutical composition of the disclosure can be formulated as a soft gelatin capsules in 2 strengths: 1) about 70 mg/mL solution of the compound in PEG 400; or 2) about 280 mg/mL suspension of the compound in PEG 400 and PEG 4000. The 70 mg capsules contain 1 mL of a 70 mg/mL solution of a compound or a pharmaceutical composition and can be, for example, clear, transparent, and oblong. The 280 mg capsules contain 1 mL of a 280 mg/mL solution of a compound or pharmaceutical composition and can be, for example, of an opaque yellow-orange color.

### Dosing

The present disclosure provides a maximally tolerated dose (MTD) of a compound disclosed herein in combination with a checkpoint inhibitor.

In some embodiments, the individual dose administered to a subject can be in an amount of from about 1 mg to about 3000 mg. In some embodiments, the individual dose administered to a subject can be in an amount of from about 1 mg to about 250 mg, from about 250 mg to about 500 mg, from about 500 mg to about 750 mg, from about 750 mg to about 1000 mg, from about 1000 mg to about 1250 mg, from about 1250 mg to about 1500 mg, from about 1500 mg to about 1750 mg, from about 1750 mg to about 2000 mg, from about 2000 mg to about 2250 mg, from about 2250 mg to about 2500 mg, from about 2500 mg to about 2750 mg, or from about 2750 mg to about 3000 mg.

In some embodiments, the individual dose administered to a subject can be in an amount of at least about 1 mg, at least about 2 mg, at least about 3 mg, at least about 4 mg, at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 125 mg, at least about 150 mg, at least about 175 mg, at least about 200 mg, at least about 250 mg, at least about 280 mg, at least about 300 mg, at least about 350 mg, at least about 400 mg, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, at least about 1000 mg, at least about 1050 mg, at least about 1100 mg, at least about 1150 mg, at least about 1200 mg, at least about 1250 mg, at least about 1300 mg, at least about 1350 mg, at least about 1400 mg, at least about 1450 mg, at least about 1500 mg, at least about 1550 mg, at least about 1600 mg, at least about 1650 mg, at least about 1700 mg, at least about 1750 mg, at least about 1800 mg, at least about 1850 mg, at least about 1900 mg, at least about 1950 mg, at least about 2000 mg, at least about 2050 mg, at least about 2100 mg, at least about 2150 mg, at least about 2200 mg, at least about 2250 mg, at least about 2300 mg, at least about 2350 mg, at least about 2400 mg, at least about 2450 mg, at least about 2500 mg, at least about 2550 mg, at least about 2600 mg, at least about 2650 mg, at least about 2700 mg, at least about 2750 mg, at least about 2800 mg, at least about 2850 mg, at least about 2900 mg, at least about 2950 mg, or at least about 3000 mg. In some embodiments, the individual dose administered to a subject can be in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 280 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200 mg, about 2250 mg, about 2300 mg, about 2350 mg, about 2400 mg, about 2450 mg, about 2500 mg, about 2550 mg, about 2600 mg, about 2650 mg, about 2700 mg, about 2750 mg, about 2800 mg, about 2850 mg, about 2900 mg, about 2950 mg, or about 3000 mg. In some embodiments, the individual dose administered to a subject can be in an amount of at most about 1 mg, at most about 2 mg, at most about 3 mg, at most about 4 mg, at most about 5 mg, at most about 10 mg, at most about 15 mg, at most about 20 mg, at most about 25 mg, at most about 30 mg, at most about 35 mg, at most about 40 mg, at most about 45 mg, at most about 50 mg, at most about 55 mg, at most about 60 mg, at most about 65 mg, at most about 70 mg, at most about 75 mg, at most about 80 mg, at most about 85 mg, at most about 90 mg, at most about 95 mg, at most about 100 mg, at most about 125 mg, at most about 150 mg, at most about 175 mg, at most about 200 mg, at most about 250 mg, at most about 280 mg, at most about 300 mg, at most about 350 mg, at most about 400 mg, at most about 450 mg, at most about 500 mg, at most about 550 mg, at most about 600 mg, at most about 650 mg, at most about 700 mg, at most about 750 mg, at most about 800 mg, at most about 850 mg, at most about 900 mg, at most about 950 mg, at most about 1000 mg, at most about 1050 mg, at most about 1100 mg, at most about 1150 mg, at most about 1200 mg, at most about 1250 mg, at most about 1300 mg, at most about 1350 mg, at most about 1400 mg, at most about 1450 mg, at most about 1500 mg, at most about 1550 mg, at most about 1600 mg, at most about 1650 mg, at most about 1700 mg, at most about 1750 mg, at most about 1800 mg, at most about 1850 mg, at most about 1900 mg, at most about 1950 mg, at most about 2000 mg, at most about 2050 mg, at most about 2100 mg, at most about 2150 mg, at most about 2200 mg, at most about 2250 mg, at most about 2300 mg, at most about 2350 mg, at most about 2400 mg, at most about 2450 mg, at most about 2500 mg, at most about 2550 mg, at most about 2600 mg, at most about 2650 mg, at most about 2700 mg, at most about 2750 mg, at most about 2800 mg, at most about 2850 mg, at most about 2900 mg, at most about 2950 mg, or at most about 3000 mg.

In some embodiments, the individual dose administered to a subject can be in 280 mg increments. In some embodiments, the individual dose administered to a subject can be in an amount of at least about 280 mg. In some embodiments, the individual dose administered to a subject can be in an amount of at least about 560 mg. In some embodiments, the individual dose administered to a subject can be in an amount of at least about 840 mg. In some embodiments, the individual dose administered to a subject can be in an amount of at least about 1120 mg. In some embodiments, the individual dose administered to a subject can be in an amount of at least about 1200 mg. In some embodiments, the individual dose administered to a subject can be in an amount of at least about 1400 mg. In some embodiments, the individual dose administered to a subject can be in an amount of at least about 1500 mg. In some embodiments, the individual dose administered to a subject can be in an amount of at least about 1680 mg. In some embodiments, the individual dose administered to a subject can be in an amount of at least about 1800 mg. In some embodiments, the individual dose administered to a subject can be in an amount of at least about 1960 mg. In some embodiments, the individual dose administered to a subject can be in an amount of at least about 2000 mg. In some embodiments, the individual dose administered to a subject can be in an amount of at least about 2200 mg. In some embodiments, the individual dose administered to a subject can be in an amount of at least about 2240 mg. In some embodiments, the individual dose administered to a subject can be in an amount of at least about 2500 mg. In some embodiments, the individual dose administered to a subject can be in an amount of at least about 2520 mg. In some embodiments, the individual dose administered to a subject can be in an amount of at least about 2800 mg. In some embodiments, the individual dose administered to a subject can be in an amount of at least about 3000 mg. In some embodiments, the individual dose administered to a subject can be in an amount of at least about 3080 mg. In some embodiments, the individual dose administered to a subject can be in an amount of about 280 mg. In some embodiments, the individual dose administered to a subject can be in an amount of about 560 mg. In some embodiments, the individual dose administered to a subject can be in an amount of about 840 mg. In some embodiments, the individual dose administered to a subject can be in an amount of about 1120 mg. In some embodiments, the individual dose administered to a subject can be in an amount of about 1200 mg. In some embodiments, the individual dose administered to a subject can be in an amount of about 1500 mg. In some embodiments, the individual dose administered to a subject can be in an amount of about 1800 mg. In some embodiments, the individual dose administered to a subject can be in an amount of about 2000 mg. In some embodiments, the individual dose administered to a subject can be in an amount of about 2200 mg. In some embodiments, the individual dose administered to a subject can be in an amount of about 2500 mg. In some embodiments, the individual dose administered to a subject can be in an amount of about 2800 mg. In some embodiments, the individual dose administered to a subject can be in an amount of about 3000 mg. In some embodiments, the individual dose administered to a subject can be in an amount of at most about 280 mg. In some embodiments, the individual dose administered to a subject can be in an amount of at most about 560 mg. In some embodiments, the individual dose administered to a subject can be in an amount of at most about 840 mg. In some embodiments, the individual dose administered to a subject can be in an amount of at most about 1120 mg. In some embodiments, the individual dose administered to a subject can be in an amount of at most about 1200 mg. In some embodiments, the individual dose administered to a subject can be in an amount of at most about 1500 mg. In some embodiments, the individual dose administered to a subject can be in an amount of at most about 1800 mg. In some embodiments, the individual dose administered to a subject can be in an amount of at most about 2000 mg. In some embodiments, the individual dose administered to a subject can be in an amount of at most about 2200 mg. In some embodiments, the individual dose administered to a subject can be in an amount of at most about 2500 mg. In some embodiments, the individual dose administered to a subject can be in an amount of at most about 2800 mg. In some embodiments, the individual dose administered to a subject can be in an amount of at most about 3000 mg.

In some embodiments, a compound described herein can be administered to a subject in an amount of about 0.1 mg/kg to about 500 mg/kg, about 1 mg/kg to about 500 mg/kg, about 0.1 mg/kg to about 300 mg/kg, about 1 mg/kg to about 300 mg/kg, or about 0.1 mg/kg to about 30 mg/kg. In some embodiments, the compound disclosed herein is administered to a subject in an amount of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 120 mg/kg, about 150 mg/kg, about 160 mg/kg, about 180 mg/kg, about 200 mg/kg, about 240 mg/kg, about 250 mg/kg, about 300 mg/kg, about 350 mg/kg, about 360 mg/kg, about 400 mg/kg, about 450 mg/kg, about 500 mg/kg, or about 600 mg/kg of the subject.

In some embodiments, dose escalation can be implemented in 70 mg increments to arrive at an effective dose to treat a condition disclosed herein. In some embodiments, a dose disclosed herein with a maximum biological effect can be administered to a subject in need thereof.

In some embodiments, a compound of the disclosure is administered intravenously. In some embodiments, a compound of the disclosure can be administered for about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 consecutive days. In some embodiments, a compound of the disclosure can be administered for about 7 consecutive days. In some embodiments, a compound of the disclosure can be administered for about 14 consecutive days. In some embodiments, a compound of the disclosure can be administered for about 21 consecutive days. In some embodiments, a compound of the disclosure can be administered for about 28 consecutive days.

In some embodiments, a compound of the disclosure can be administered for about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28 consecutive days of a 28-day cycle. In some embodiments, a compound of the disclosure can be administered for about 7 consecutive days of a 28-day cycle. In some embodiments, a compound of the disclosure can be administered for about 14 consecutive days of a 28-day cycle. In some embodiments, a compound of the disclosure can be administered for about 21 consecutive days of a 28-day cycle. In some embodiments, a compound of the disclosure can be administered for about 28 consecutive days of a 28-day cycle.

A compound disclosed herein can be administered via subcutaneous or intravenous injection. The volume of an injection can be about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 2.1 mL, about 2.2 mL, about 2.3 mL, about 2.4 mL, about 2.5 mL, about 2.6 mL, about 2.7 mL, about 2.8 mL, about 2.9 mL, or about 3 mL.

In some embodiments, a compound of the disclosure can be administered at a dose of 560 mg twice a day. In some embodiments, a compound of the disclosure can be administered at a dose of 840 mg twice a day. In some embodiments, a compound of the disclosure can be administered at a dose of 1120 mg twice a day. In some embodiments, a compound of the disclosure can be administered at a dose of 1400 mg twice a day. In some embodiments, a compound of the disclosure can be administered at a dose of 1680 mg twice a day. In some embodiments, a compound of the disclosure can be administered at a dose of 1960 mg twice a day. In some embodiments, a compound of the disclosure can be administered at a dose of 2240 mg twice a day. In some embodiments, a compound of the disclosure can be administered at a dose of 2520 mg twice a day. In some embodiments, a compound of the disclosure can be administered at a dose of 2800 mg twice a day. In some embodiments, a compound of the disclosure can be administered at a dose of 3080 mg twice a day.

In some embodiments, a compound of the disclosure can be administered at a dose of 560 mg split in two doses throughout a day. In some embodiments, a compound of the disclosure can be administered at a dose of 840 mg split in two doses throughout a day. In some embodiments, a compound of the disclosure can be administered at a dose of 1120 mg split in two doses throughout a day. In some embodiments, a compound of the disclosure can be administered at a dose of 1400 mg split in two doses throughout a day. In some embodiments, a compound of the disclosure can be administered at a dose of 1680 mg split in two doses throughout a day. In some embodiments, a compound of the disclosure can be administered at a dose of 1960 mg split in two doses throughout a day. In some embodiments, a compound of the disclosure can be administered at a dose of 2240 mg split in two doses throughout a day. In some embodiments, a compound of the disclosure can be administered at a dose of 2520 mg split in two doses throughout a day. In some embodiments, a compound of the disclosure can be administered at a dose of 2800 mg split in two doses throughout a day. In some embodiments, a compound of the disclosure can be administered at a dose of 3080 mg split in two doses throughout a day.

In some embodiments, a compound of the disclosure can be administered at a first dose in the morning or early afternoon, and a second dose in the afternoon or evening. In some embodiments, the first dose is about 280 mg, about 560 mg, about 840 mg, about 1120 mg, about 1400 mg, about 1680 mg, about 1960 mg, about 2240 mg, about 2520 mg, about 2800 mg, or about 3080 mg. In some embodiments, the second dose is about 280 mg, about 560 mg, about 840 mg, about 1120 mg, about 1400 mg, about 1680 mg, about 1960 mg, about 2240 mg, about 2520 mg, about 2800 mg, or about 3080 mg.

In some embodiments, a compound of the disclosure can be administered at a dose of about 800 mg for 2 to 5 days every other week. In some embodiments, a compound of the disclosure can be administered at a dose of from about 650 mg to about 1700 mg for 3 to 6 days every other week. In some embodiments, a compound of the disclosure can be administered at a dose of from about 800 mg to about 1500 mg for 2 days every week for 3 weeks, followed by 1 week without treatment. In some embodiments, a compound of the disclosure can be administered at a dose of about 1800 mg per day for 3 days every other week. In some embodiments, a compound of the disclosure can be administered at a dose of about 70 mg, about 140 mg, about 280 mg, about 560 mg, or about 700 mg once a day for one week. In some embodiments, a compound of the disclosure can be administered at a dose of about 70 mg, about 140 mg, about 280 mg, about 560 mg, about 700 mg, or about 1120 mg per day twice a day for the first 14 days of a 21 day cycle.

In some embodiments, a compound of the disclosure can be administered at a dose of about 560 mg once in the morning and about 280 mg in the evening for 2 weeks, followed by one week off. In some embodiments, the compound is administered in the fasting state, about 1-2 hours before or after a meal.

A dosing regimen for intravenous administration of a compound can be, for example, 1,800 mg/day, given on days 1-3 of a 14-day cycle for 8 cycles and then every on days 1-3 of a 28-day cycle thereafter. In combination with a dose of nivolumab, a 3+3 dose escalation can be incorporated to find the RP2D (recommended phase 2 dose). Dose escalation cohorts can receive the full dose of nivolumab and initially reduced doses of rigosertib, for example, 1,200 mg/day, then 1500 mg/day, then 1800 mg/day given on days 1-3 of a 14-day cycle for 8 cycles and then every on days 1-3 of a 28-day cycle thereafter. In solid tumor studies, where there is less risk of bone marrow toxicity, the dose can be further incrementally increased to 2,100 mg/day, 2,400 mg/day until the MTD and RP2D are established according to 3+3 dose escalation.

In some embodiments, a compound of the disclosure can be administered in a first dose of about 840 mg administered approximately 1-2 hours before breakfast, followed by a second dose of about 280 mg administered about 2 hours after lunch or about 6-8 hours after the first dose. In some embodiments, a compound of the disclosure can be administered in a first dose of about 560 mg administered approximately 1-2 hours before breakfast, followed by a second dose of about 280-560 mg administered about 2 hours after lunch or about 6-8 hours after the first dose.

A dosing regimen disclosed herein can be, for example, one dose of 840 mg of oral rigosertib in the morning and 560 mg in the afternoon. Dose escalation can continue, depending on the observed number of dose limiting toxicities (DLT). A dosing regimen disclosed herein can be, for example, 840 mg twice daily, then 1,120 mg in the morning and then 840 mg in the afternoon. Dose escalation can continue by, for example, 280 mg increments. For example, a morning dose can be increased by 280 mg, and then the afternoon dose can be increased by 280 mg.

A dose escalation can continue until two or more DLTs are observed in a single cohort. At that point, a prior dose cohort can be expanded to 6 patients and if less than 2 DLTs occur in those 6 patients, then that dose can be considered the MTD.

Alternatively, the dose escalation can occur in 70 mg increments. Additionally, a much larger percentage of the total daily dose can be administered in the morning dose and a lower percentage in the afternoon dose (i.e. 1,400 mg in the a.m. and 560 mg in the afternoon).

In some embodiments, a subject is encouraged to drink at least 2 L of fluids per day. In some embodiments, a subject is advised to take 650 mg sodium bicarbonate three times per day, particularly if the urine pH is less than about 7.5.

### Methods of Administration

A compound described herein can be administered before, during, or after the occurrence of a disease or condition, and the timing of administering the composition containing a compound can vary. For example, a compound can be used as a prophylactic and can be administered continuously to subjects with a propensity to conditions or diseases to lessen or reduce a likelihood of the occurrence of the disease or condition. A compound and composition can be administered to a subject during or as soon as possible after the onset of the symptoms. The administration of a compound can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years. The length of treatment can vary for each subject.

In some embodiments, a compound of the disclosure can be administered once a day. In some embodiments, a compound of the disclosure can be administered twice a day. In some embodiments, a compound of the disclosure can be administered three times a day. In some embodiments, compound absorption can be interfered by food. In some embodiments, the dosing is separated from a meal by at least about 15 min, at least about 30 min, at least about 45 min, at least about 1 hr, at least about 1.5 hr, at least about 2 hr, at least about 2.5 hr, at least about 3 hr, at least about 3.5 hr, at least about 4 hr, at least about 4.5 hr, at least about 5 hr, at least about 5.5 hr, or at least about 6 hr. In some embodiments, the dosing is separated from a meal by at least about 30 min. In some embodiments, the dosing is separated from a meal by at least about 1 hr. In some embodiments, the dosing is separated from a meal by at least about 2 hr. In some embodiments, the compound is administered before the subject consumes food. In some embodiments, the compound is administered after the subject consumes food. In some embodiments, the compound is administered before the subject consumes a drink. In some embodiments, the compound is administered after the subject consumes a drink.

In some embodiments, the morning dose of a compound is taken after an overnight fast and an hour before breakfast. In some embodiments, an afternoon dose of a compound is taken 2 hours after lunch and an hour before dinner or other food. In some embodiments, a first therapeutically-effective amount of the compound is administered to the subject in a morning of a day, and a second therapeutically-effective amount of the compound is administered to the subject in an afternoon of the day. In some embodiments, a first therapeutically-effective amount of the compound is administered to the subject in a morning of a day, and a second therapeutically-effective amount of the compound is administered to the subject in an evening of the day. In some embodiments, the first therapeutically-effective amount and the second therapeutically-effective amount are the same. In some embodiments, the first therapeutically-effective amount and the second therapeutically-effective amount are different. In some embodiments, the first therapeutically-effective amount is greater than the second therapeutically-effective amount. In some embodiments, the first therapeutically-effective amount is lesser than the second therapeutically-effective amount.

Intravenous rigosertib can be given through a large bore intravenous catheter to avoid the risks of extravasation. The IV infusion can continuous (CIV) for 72 hours with three separate infusion bags delivered for 24 hours each via a standard infusion pump.

To mitigate a risk of a genitourinary adverse event, a second dose of a compound disclosed herein can be administered in the afternoon, a subject disclosed herein can be instructed to hydrate orally prior to bedtime, a subject disclosed herein can be instructed to empty the bladder prior to bedtime, or any combination thereof.

The urine pH of a subject disclosed herein can be monitored. If the urine pH is acidic, the subject can be administered oral bicarbonate. A urine dipstick obtained from a subject disclosed herein can further be monitored for evidence of microscopic hematuria.

A dosing schedule for administration of a compound described herein include, but are not limited to, once daily (QD), twice daily (BID), three times daily (TID), four times daily (QID), once weekly, twice weekly, three times weekly, once monthly, twice monthly, and once every other month. In some embodiments, a compound of the disclosure is administered once daily. In some embodiments, a compound of the disclosure is administered twice daily. In some embodiments, a compound of the disclosure is administered three times daily. In some embodiments, a compound of the disclosure is administered four times daily.

An approximate dose can be predicted or determined on the basis of data existing in other species. In some embodiments, allometric scaling can be used to exchange a drug dose based on normalization of dose to body surface area. Allometric scaling considers the sizes of individual species based on body surface area, which is related to metabolic rate of an animal that is established through evolutionary adaptation of animals to their size. A no observed adverse effect level (NOAEL) is first determined in an animal species, the NOAEL is converted to a human equivalent dose (HED), an appropriate animal species is selected, a safety factor is applied, and a pharmacologically active dose is determined.

NOAEL, the highest dose level that does not cause significant adverse effects, is a typical index for safety obtained from animal experiments to determine a safe starting dose.

NOAEL values can be converted to HED on the basis of the body surface correction factor using appropriate scaling factors from animal species. TABLE 1 lists HED calculation guidelines based on body surface areas. HED is determined using the equation:

HED (mg/kg)=Animal NOAEL (mg/kg)×(Weightₐₙᵢₘₐₗ [kg]/Weightₕᵤₘₐₙ[kg])^{(1-0.67)}

The HED is divided by a factor value of 10 to increase safety of the first human dose. The safety factor is accountable for differences in physiological and biological processes between human and animal species.

The correction factor (Kₘ) is estimated by dividing the average body weight (kg) of a species to its body surface area (m²). The Kₘ factor values of various animal species of TABLE 1 is used to estimate the HED as:

HED (mg/kg)=Animal doses (mg/kg)×(Animal Kₘ/Human Kₘ); or

HED (mg/kg)=Animal doses (mg/kg)×Kₘ ratio

TABLE 1

| | | | | To convert | | |
|---|---|---|---|---|---|---|
| | | | | dose in mg/kg | To convert animal dose in | |
| | Reference | Working | Body | to dose in | mg/kg to HED in mg/kg, either | |
| | body | weight | surface | mg/m², | Divide animal | Multiply animal |
| Species | weight (kg) | range (kg) | area (m²) | multiply by Kₘ | by dose | by dose |
| Human | 60 | — | 1.62 | 37 | — | — |
| Mouse | 0.02 | 0.011-0.034 | 0.007 | 3 | 12.3 | 0.081 |
| Hamster | 0.08 | 0.047-0.157 | 0.016 | 5 | 7.4 | 0.135 |
| Rat | 0.15 | 0.08-0.27 | 0.025 | 6 | 6.2 | 0.162 |
| Ferret | 0.30 | 0.16-0.54 | 0.043 | 7 | 5.3 | 0.189 |
| Guinea Pig | 0.40 | 0.208-0.700 | 0.05 | 8 | 4.6 | 0.216 |
| Rabbit | 1.8 | 0.90-3.0 | 0.15 | 12 | 3.1 | 0.324 |
| Dog | 10 | 5-17 | 0.50 | 20 | 1.8 | 0.541 |
| Monkeys | 3 | 1.4-4.9 | 0.25 | 12 | 3.1 | 0.324 |
| (rhesus) | | | | | | |
| Marmoset | 0.35 | 0.14-0.72 | 0.06 | 6 | 6.2 | 0.162 |
| Squirrel | 0.60 | 0.29-0.97 | 0.09 | 7 | 5.3 | 0.189 |
| Monkey | | | | | | |
| Baboon | 12 | 7-23 | 0.60 | 20 | 1.8 | 0.541 |
| Micro pig | 20 | 10-33 | 0.74 | 27 | 1.4 | 0.730 |
| Mini pig | 40 | 25-64 | 1.14 | 35 | 1.1 | 0.946 |

TABLE 2 provides animal equivalent dose (AED) calculation guidelines based on G body surface area. The animal equivalent dose (AED) can also be calculated on the basis of body surface area by either dividing or multiplying the human dose (mg/kg) by the Kₘ ratio provided in TABLE 2. AED can be calculated using the equation:

AED (mg/kg)=Human doses (mg/kg)×Kₘ ratio

TABLE 2

| | | | To convert human | |
|---|---|---|---|---|
| | | | dose in mg/kg to | |
| | Reference | To convert | AED in mg/kg, either | |
| | body | dose in mg/kg | Multiply | Divide |
| | weight | to dose in mg/m², | human | human |
| Species | (kg) | divide by Kₘ | dose by | dose by |
| Human | 60 | 37 | — | — |
| Mouse | 0.02 | 3 | 12.3 | 0.081 |
| Hamster | 0.08 | 5 | 7.4 | 0.135 |
| Rat | 0.15 | 6 | 6.2 | 0.162 |
| Ferret | 0.30 | 7 | 5.3 | 0.189 |
| Guinea Pig | 0.40 | 8 | 4.6 | 0.216 |
| Rabbit | 1.8 | 12 | 3.1 | 0.324 |
| Dog | 10 | 20 | 1.8 | 0.541 |
| Monkeys (rhesus) | 3 | 12 | 3.1 | 0.324 |
| Marmoset | 0.35 | 6 | 6.2 | 0.162 |
| Squirrel Monkey | 0.60 | 7 | 5.3 | 0.189 |
| Baboon | 12 | 20 | 1.8 | 0.541 |
| Micro pig | 20 | 27 | 1.4 | 0.730 |
| Mini pig | 40 | 35 | 1.1 | 0.946 |

For parenteral administration, HED conversion (mg/kg) is also based on body surface area normalization. The conversion can be made by dividing the NOAEL in appropriate species by the conversion factor. TABLE 3 provides guidelines for maximum injection volume, by species, site location, and gauge size. Injection volume of parenteral formulation is calculated by the following equation:

Injection volume (mL)=[Animal weight (kg)×Animal doses (mg/kg)]/Concentration (mg/kg)

TABLE 3

| | Route | | | | | | | | |
|---|---|---|---|---|---|---|---|---|---|
| | Subcutaneous | | | Intramuscular | | | Intraperitoneal | | |
| | Max | | | Max | | | Max | | |
| | injection | | Gauge | injection | | Gauge | injection | | Gauge |
| Species | volume (mL) | Site | size | volume (mL) | Site | size | volume (mL) | Site | size |
| Human | <2 | Upper arm, | 25-31 | 2-5 | Deltoid, vastus | 21-23 | 2-5 | Peritoneum | — |
| | | abdomen, thigh, | | | lateralis | | | | |
| | | buttock | | | | | | | |
| Mouse | 1-2 | Back (scruff), | <20 | <0.1 | Quadriceps, | 25 | 2-3 | Lateral | 25 |
| | | lower abdomen | | | posterior thigh | | | tail vein | |
| Hamster | 3-4 | Dorsum between | <20 | <0.1 | hind limb, | 25 | 2-3 | Lower left | 25-56 |
| | | scapula | | | caudal thigh | | | quadrant | |
| Rat | 5-10 | Back (scruff), | <20 | <0.3 | quadriceps, | 25-56 | 5-10 | Lower left | 24 |
| | | lower abdomen | | | hamstrings | | | quadrant | |
| Dog | 100-200 | Dorsum between | 22 | 2-5 | hind limb, | 22-25 | 100-200 | Cephalic, | 22-25 |
| | | scapula | | | caudal thigh | | | saphenous | |
| Guinea pig | 5-10 | Dorsum between | 25 | 0.3 | Lind limb, | 25 | 10-15 | Lower left | 25 |
| | | scapula | | | caudal thigh, | | | quadrant | |
| | | | | | lumbar muscles | | | | |
| Rabbit | 10-50 | Dorsum | 22-25 | <0.5 | Hind limb, | 22-25 | 50-100 | Lower left | 22 |
| | | | | | caudal thigh | | | quadrant | |
| Monkey | 10-30 | Dorsum | 22-23 | 1-3 | Hind limb, | 22-23 | 25-50 | Peritoneum | 20 |
| (Rhesus) | | | | | caudal thigh | | | | |
| Squirrel | 5-10 | Scuff | 20 | 1-3 | Quadriceps, | 20 | 25-50 | Peritoneum | 20 |
| Monkey | | | | | posterior | | | | |
| | | | | | thigh, triceps | | | | |

| | Intravenous | | | Intradermal | | |
|---|---|---|---|---|---|---|
| | Max | | | Max | | |
| | injection | | Gauge | injection | | Gauge |
| Species | volume (mL) | Site | size | volume (mL) | Site | size |
| Human | <250 | Vein | 18-20 | <0.10 | Dermis | 25-26 |
| Mouse | <0.2 | Lateral tail | 25 | <0.05 | Lateral | — |
| | | vein | | | abdomen | |
| Hamster | <0.2 | Lateral tarsal | 26 | <0.05 | — | — |
| | | vein, cephalic/ | | | | |
| | | lingual vein | | | | |
| Rat | <0.5 | Lateral tail/ | 24-27 | <0.05 | Lateral | — |
| | | saphenous vein | | | abdomen | |
| Dog | <100 | Cephalic/ | 21 | — | — | — |
| | | saphenous vein | | | | |
| Guinea pig | 10-15 | Lateral | 26-27 | — | Dorsum along | 25 |
| | | saphenous vein | | | flank | |
| Rabbit | <5 | Marginal ear | 25 | <0.1 | Dorsum along | 25 |
| | | vein | | | flank | |
| Monkey | 5-10 | Saphenous vein | 22-23 | — | — | — |
| (Rhesus) | | | | | | |
| Squirrel | 0.5-1 | Femoral vein | 21 | — | — | — |
| Monkey | | | | | | |

### Checkpoint Inhibitors

In some embodiments, disclosed herein is a method of treating a cancer by administering an immunomodulatory agent. In some embodiments, disclosed herein is a method of treating a cancer by administering a compound of a disclosure and a cancer immunotherapy. In some embodiments, the cancer immunotherapy targets an immune checkpoint. In some embodiments, the cancer immunotherapy can block an inhibitory checkpoint and restore immune system function. In some embodiments, disclosed herein is a method of treating a cancer by administering a compound of the disclosure and an antibody. In some embodiments, disclosed herein is a method of treating a cancer by administering a compound of the disclosure and a biologic.

Immune checkpoints are co-stimulatory and inhibitory elements intrinsic to the immune system. Immune checkpoints aid in maintaining self-tolerance and modulating the duration and amplitude of physiological immune responses to reduce injury to tissues when the immune system responds to pathogenic infection. An immune response can also be initiated when a T-cell recognizes antigens that are unique to a tumor cell (e.g. non-self antigens or tumor neo-antigens) or are characteristic of a tumor cell (e.g. tumor-associated antigens (TAAs)). The equilibrium between the co-stimulatory and inhibitory signals used to control the immune response from T-cells can be modulated by immune checkpoint proteins. After T-cells mature and activate in the thymus, T-cells can travel to sites of inflammation and injury to perform repair functions. T-cell function can occur either via direct action or through the recruitment of cytokines and membrane ligands involved in the immune system. The steps involved in T-cell maturation, activation, proliferation, and function can be regulated through co-stimulatory and inhibitory signals, namely through immune checkpoint proteins. Tumors can dysregulate checkpoint protein function as an immune-resistance mechanism. Thus, the development of modulators of checkpoint proteins can have therapeutic value. Non-limiting examples of immune checkpoint molecules include LAG3, BTLA, KIR, CTLA4, ICOS, TIM3, A2aR, PD-1, PD-L1, PD-L2, CD40L, OX40L, CD137L, CD47, B7-H3, and B7-H4. These checkpoint molecules can operate upstream of IL-2 in a pathway.

Checkpoint molecules guard against unwanted and harmful self-directed activation of the immune system (autoimmunity). Although necessary in aiding in the suppression of autoimmunity, these molecules can hinder immunotherapies aimed at targeting malignant self-cells that largely display the same array of surface molecules as the parent cells. Therapies aimed at overcoming these mechanisms of peripheral tolerance, particularly by blocking the inhibitory checkpoints, offer the potential to generate antitumor activity, either as monotherapies or in combination with other therapies that directly or indirectly enhance presentation of tumor epitopes to the immune system.

Immunological checkpoints can be molecules that regulate inhibitory signaling pathways (for example, LAG3, CTLA4, PD-1, and TIM3) or molecules that regulate stimulatory signaling pathways (for example, by ICOS). Several proteins in the extended immunoglobulin superfamily can be ligands for immunological checkpoints. Non-limiting examples of immune checkpoint ligand proteins include B7-H4, ICOSL, PD-L1, PD-L2, CD40L, OX40L, CD86, and CD137L.

In some embodiments, the checkpoint inhibitor is a cell-surface checkpoint inhibitor. In some embodiments, the checkpoint inhibitor is a CTLA-4 inhibitor, a PD-1 inhibitor, or a PD-L1 inhibitor. In some embodiments, the checkpoint inhibitor is an intracellular checkpoint inhibitor. In some embodiments, the checkpoint inhibitor is cytokine-inducible SH2-containing protein (CISH).

PD-1 is an inhibitory receptor belonging to the CD28/CTLA4 family and is expressed on the surface of activated T lymphocytes, B cells, monocytes, DCs, NK cells, and T_{regs}. In contrast to CTLA4, the major role of PD-1 is limitation of activity of T cells in peripheral tissues at the time of an inflammatory response to infection and to limit autoimmunity. Chronic antigen exposure can lead to persistently-high levels of PD-1 expression. This expression can induce a state of exhaustion or anergy of antigen-specific T-cells. This state can be at least partially reversed by PD-1 blockade.

Two ligands for PD-1, PD-L1 and PD-L2, are expressed on T cells, APCs, and malignant cells. The ligands function to suppress self-reactive lymphocytes and to inhibit the effector function of TAA-specific cytotoxic T lymphocytes (CTLs). Accordingly, a therapy that targets PD-1, PD-L1, or PD-L2 has the potential to restore the cytotoxic activity of TAA-specific T cells.

Upon engagement of ligands, PD-1 can inhibit kinases involved in T-cell activation through the phosphatase, SHP2. PD-1 can limit the activity of T-cells in peripheral tissues at the time of an inflammatory response to an infection and limit autoimmunity. The decrease in the proliferation of T-cells can lead to a decrease in IL-2 secretion. PD-1 can also be highly expressed on T_{regs}, which can have an immunosuppressive function, and further increase the proliferation of T_{regs}. Tumors can be highly infiltrated with T_{regs}; thus, blockade of PD-1 can diminish the immunosuppressive function of the intratumoral T_{regs}.

Due to the broad expression pattern, PD-1 can also enhance NK activity in tumors or tissues. PD-1 can increase antibody production through PD-1V B-cells. Chronic antigen exposure observed in viral infection and cancer can lead to persistent PD-1 activation and T-cell anergy among cognate antigen-specific T-cells. This anergic state can be reversed through a blockade of PD-1.

PD-1 can also be expressed on tumor infiltrating lymphocytes (TILs) in many tumor types. The enhanced PD-1 expression of CD4⁺ cells can reflect the high expression of PD-1 on regulatory T-cells within tumors. PD-1 can also be highly expressed on CD8⁺ cells and can reflect an anergic state. Consistent with the increased expression of PD-1 on lymphocytes from many tumors, the ligands of PD-1 can also be highly expressed on the tumor cell surface. PD-L1 can be highly expressed on, for example, melanoma, ovarian cancer, lung cancer, and renal cancer cells. PD-L2 can be highly expressed on, for example, primary mediastinal B-cell lymphoma, follicular cell B-cell lymphoma, and Hodgkin's lymphoma. Anti-PD-1 antibodies can induce regression of several tumor types including colon, renal, lung, and melanoma. Therapies targeting PD-1 directly or the interaction between PD-1 and a ligand include nivolumab, pembrolizumab, pidilizumab, and AMP-224.

In some embodiments, the anti-PD-1 antibody is Pembrolizumab. In some embodiments, the anti-PD-1 antibody is Nivolumab. In some embodiments, the anti-PD-1 antibody is Pidilizumab. Non-limiting examples of anti-PD-1 antibodies include AGEN-2034, AMP-224, BCD-100, BGBA-317, BI-754091, CBT-501, CC-90006, cemiplimab, durvalumab+MEDI-0680, GLS-010, IBI-308, JNJ-3283, JS-001, MEDI-0680, MGA-012, MGD-013, pazopanib hydrochloride+pembrolizumab, PDR-001, PF-06801591, REGN-2810, SHR-1210, TSR-042, LZM-009, and ABBV-181.

In some embodiments, the anti-PD-L1 antibody is, for example, Durvalumab, Atezolizumab, Avelumab, CX-072, BMS-936559, SHR-1316, M-7824, LY-3300054, FAZ-053, KN-035, CA-170, CK-301, CS-1001, HLX-10, MCLA-145, MSB-2311, or MEDI-4736.

LAG3 (Lymphocyte-activation gene 3) is expressed on activated antigen-specific cytotoxic T cells and can enhance the function of regulatory T-cells and independently inhibit CD8+ effector T-cell activity. LAG3 is a CD-4-like negative regulatory protein with a high affinity binding site to MHC Class II, which is upregulated on some epithelial cancers, to provide tolerance of T cell proliferation and homeostasis. Blockage of the LAG-3/Class II interaction using a LAG-3-IG fusion protein enhances antitumor immune responses. Therapeutics targeting LAG3 include IMP321 and other monoclonal antibodies.

BTLA (B- and T-lymphocyte attenuator) can inhibit T-cells when associated with HVEM (herpes-virus entry mediator) as a ligand. HVEM can be expressed on melanoma and endothelial cancer cells. BTLA levels can be high on TILs from subjects with melanoma, and BTLA-expressing T-cells can be inhibited in the presence of HVEM.

KIR (killer immunoglobulin-like receptor) is expressed by Natural Killer (NK) cells and a subset of T lymphocytes. KIRs are largely cell surface inhibitory receptors specific for allelic forms of human leukocyte antigen (HLA) class I molecules. Upon engagement with HLA class I molecules, KIRs block NK cell activation and function. Blockage of KIRs can lead to blockage of NK cell activation and function.

CTLA4 (cytotoxic T-lymphocyte antigen 4) is also known as CD152 (Cluster of differentiation 152). CTLA4 shares sequence homology and ligands (CD80/B7-1 and CD86/B7-2) with the costimulatory molecule CD28, but differs by delivering inhibitory signals to T cells expressing CTLA4 as a receptor. CTLA4 has a much higher overall affinity for both ligands and can out-compete CD28 for binding when ligand densities are limiting.

CTLA4 is expressed on the surface of CD8+ effector T-cells and plays a functional role in the initial activation stages of both naive and memory T cells. CTLA4 counteracts the activity of CD28 via increased affinity for CD80 and CD86 during the early stages of T-cell activation. The major functions of CTLA4 include downmodulation of helper T-cells and enhancement of regulatory T-cell immunosuppressive activity. Evidence for the importance of CTLA4 can be demonstrated through the lethal systemic immune hyperactivation phenotype in Ctla4−/− mice.

CTLA4 can also downregulate immune system functions via inhibition of IL-2 production and IL-2 receptor expression. CTLA4 can inhibit CD28-dependent upregulation of IL-2, and the inhibition of IL-2 production can lead to cell cycle arrest. The decrease in IL-2 and subsequent cell cycle arrest can account for the reduced T-cell proliferation observed in the presence of CTLA4.

CTLA4 includes an extracellular domain, a transmembrane domain, and a cytoplasmic tail. Alternate transcriptional splice variants encoding different isoforms exist. The membrane-bound isoform can function as a homodimer interconnected by a disulfide bond, and the soluble isoform can function as a monomer. Mutations in CTLA4 can be associated with, for example, insulin-dependent diabetes mellitus, Graves' disease, Hashimoto thyroiditis, celiac disease, systemic lupus erythematosus, thyroid-associated orbitopathy, and other autoimmune diseases.

Therapies targeting CTLA4 can be developed to circumvent tumor resistance mechanisms. Inhibition of CTLA4 can lead to a broad enhancement of immune responses that are dependent on helper T-cells. Thus, therapeutics aimed at blocking CTLA4 can assist in evading tumor cell resistance mechanisms. Examples of therapies targeting CTLA4 include ipilimumab and tremelimumab, monoclonal antibodies that can target CTLA4 and can be used in the treatment of, for example, melanoma. Therapies can also be directed toward increasing the activity of CTLA4 for the treatment of autoimmune diseases. Fusion proteins of CTLA4 and antibodies can increase the immune response in patients with, for example, rheumatoid arthritis, or patients sensitized to the Epstein Barr Virus (EPV) undergoing renal transplantation.

ICOS (Inducible T-cell COStimulator), also known as CD278, is a CD28-superfamily costimulatory molecule that is expressed on activated T cells. CD40 and CD134 are examples of additional co-stimulatory molecules.

TIM3 (T-cell immunoglobulin and mucin domain-containing protein 3), also known as Hepatitis A virus cellular receptor 2 (HAVCR2) is a Th1-specific cell surface protein that regulates macrophage activation. TIM3 can inhibit helper T-cell responses via association with the TIM3 ligand, galectin 9. Galectin 9 can be upregulated in various types of cancer, including breast cancer. TIM3 can be co-expressed with PD-1 on tumor specific CD8+ T-cells and inhibition of both molecules can significantly enhance the proliferation and cytokine production of T-cells.

Four types of adenosine receptors, A1, A2A, A2B, and A3 receptors, are expressed on the surface of immune cells. On T cells, the predominant subtype expressed is A2A adenosine receptor (A2aR). Immunosupressive signaling through the A2aR receptor can control the cytokine secretion pattern of NK cells and function to protect inflamed tissues from excessive damage by immune cells. A2aR receptors inhibit T-cell responses by driving CD4+ T-cells to express FOXP3 (forkhead box P3). FOXP3 can then cause the CD4+ T-cells to develop into regulatory T-cells. Deletion of A2aR can lead to pathological inflammatory responses to infection.

B7-H3 can costimulate proliferation of both CD4+ and CD8+ T cells, enhance the induction of cytotoxic T cells, and selectively stimulate interferon γ (IFN-γ) production in the presence of T cell receptor signaling. The B7-H3 receptor can be expressed in dendritic cells and monocytes.

B7-H4, also known as B7S1 or B7x, is a coinhibitory member of the B7 family that negatively regulates neutrophil-mediated innate immune responses. The B7-H4 receptor can be expressed in B cells and antigen presenting cells. The B7-H4 receptor can also be overexpressed in breast, ovarian, lung cancer, and other solid tumors.

Immunotherapies disclosed herein include those that modulate a checkpoint molecule or a checkpoint protein. Non-limiting examples of agents that modulate a checkpoint molecule or a checkpoint protein include cytokines, immunotoxins, recombinant proteins, antibodies, monoclonal antibodies, tumor-specific monoclonal antibodies, antibody-drug conjugates, immunotoxins, and any agent that modulates, up-regulates, down-regulates, agonizes, antagonizes, inhibits, or induces one or more of LAG3, BTLA, KIR, CTLA4, ICOS, TIM3, A2aR, PD-1, B7-H3, B7-H4, ICOSL, PD-L1, PD-L2, CD40L, OX40L, CD47, CD86, and CD137L. In some embodiments, the immunotoxin therapy is a therapy against one or more of CD5, CD7, CD19, CD22, CD25 (T-NHL), CD30, and CD38 (B-NHL). In some embodiments, the immunotherapy is ipilimumab, nivolumab, tremelimumab, pembrolizumab, pidilizumab, AMP-224 or Resimmune™ (also called A-dmDT390-bisFv(UCHT1)). The agent can be administered in the same unit dosage form as a compound described herein, or in a separate dosage form in any order or concurrently.

Non-limiting examples of cells whose activity can be modulated by a combination therapy include: endothelial cells; B cells; CD4; CD8; blood cells, including red blood cells and white blood cells; dendritic cells, including dendritic antigen presenting cells; leukocytes; macrophages; memory B cells; memory T cells; monocytes; NK cells; neutrophil granulocytes; helper T cells; and cytotoxic T cells.

In some embodiments, the checkpoint inhibitor is ipilimumab, nivolumab, pembrolizumab, atezolizumab, avelumab, durvalumab, or cemiplimab. In some embodiments, the checkpoint inhibitor is nivolumab. In some embodiments, the checkpoint inhibitor is pembrolizumab. In some embodiments, the checkpoint inhibitor is avelumab. In some embodiments, the checkpoint inhibitor is durvalumab.

In some embodiments, a checkpoint inhibitor can be administered at a dose of from about 0.1 mg/kg to about 10 mg/kg. In some embodiments, a checkpoint inhibitor can be administered at a dose of from about 0.1 mg/kg to about 0.5 mg/kg, from about 0.5 mg/kg to about 1 mg/kg, from about 1 mg/kg to about 1.5 mg/kg, from about 1.5 mg/kg to about 2 mg/kg, from about 2 mg/kg to about 2.5 mg/kg, from about 2.5 mg/kg to about 3 mg/kg, from about 3 mg/kg to about 3.5 mg/kg, from about 3.5 mg/kg to about 4 mg/kg, from about 4 mg/kg to about 4.5 mg/kg, from about 4.5 mg/kg to about 5 mg/kg, from about 5 mg/kg to about 5.5 mg/kg, from about 5.5 mg/kg to about 6 mg/kg, from about 6 mg/kg to about 6.5 mg/kg, from about 6.5 mg/kg to about 7 mg/kg, from about 7 mg/kg to about 7.5 mg/kg, from about 7.5 mg/kg to about 8 mg/kg, from about 8 mg/kg to about 8.5 mg/kg, from about 8.5 mg/kg to about 9 mg/kg, from about 9 mg/kg to about 9.5 mg/kg, or from about 9.5 mg/kg to about 10 mg/kg. In some embodiments, a checkpoint inhibitor can be administered at a dose of from about 0.1 mg/kg to about 0.5 mg/kg. In some embodiments, a checkpoint inhibitor can be administered at a dose of from about 0.5 mg/kg to about 1 mg/kg. In some embodiments, a checkpoint inhibitor can be administered at a dose of from about 1 mg/kg to about 1.5 mg/kg. In some embodiments, a checkpoint inhibitor can be administered at a dose of from about 1.5 mg/kg to about 3 mg/kg. In some embodiments, a checkpoint inhibitor can be administered at a dose of from about 3 mg/kg to about 5 mg/kg.

In some embodiments, a checkpoint inhibitor can be administered at a dose of at least about 0.1 mg/kg, at least about 0.5 mg/kg, at least about 1 mg/kg, at least about 1.5 mg/kg, at least about 2 mg/kg, at least about 2.5 mg/kg, at least about 3 mg/kg, at least about 3.5 mg/kg, at least about 4 mg/kg, at least about 4.5 mg/kg, at least about 5 mg/kg, at least about 5.5 mg/kg, at least about 6 mg/kg, at least about 6.5 mg/kg, at least about 7 mg/kg, at least about 7.5 mg/kg, at least about 8 mg/kg, at least about 8.5 mg/kg, at least about 9 mg/kg, at least about 9.5 mg/kg, or at least about 10 mg/kg. In some embodiments, a checkpoint inhibitor can be administered at a dose of about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, a checkpoint inhibitor can be administered at a dose of about 1 mg/kg. In some embodiments, a checkpoint inhibitor can be administered at a dose of about 2.5 mg/kg. In some embodiments, a checkpoint inhibitor can be administered at a dose of about 3 mg/kg. In some embodiments, a checkpoint inhibitor can be administered at a dose of about 3.5 mg/kg.

In some embodiments, a checkpoint inhibitor can be administered at a dose of at most about 0.1 mg/kg, at most about 0.5 mg/kg, at most about 1 mg/kg, at most about 1.5 mg/kg, at most about 2 mg/kg, at most about 2.5 mg/kg, at most about 3 mg/kg, at most about 3.5 mg/kg, at most about 4 mg/kg, at most about 4.5 mg/kg, at most about 5 mg/kg, at most about 5.5 mg/kg, at most about 6 mg/kg, at most about 6.5 mg/kg, at most about 7 mg/kg, at most about 7.5 mg/kg, at most about 8 mg/kg, at most about 8.5 mg/kg, at most about 9 mg/kg, at most about 9.5 mg/kg, or at most about 10 mg/kg. In some embodiments, a checkpoint inhibitor can be administered at a dose of at most about 1 mg/kg. In some embodiments, a checkpoint inhibitor can be administered at a dose of at most about 2 mg/kg. In some embodiments, a checkpoint inhibitor can be administered at a dose of at most about 3 mg/kg. In some embodiments, a checkpoint inhibitor can be administered at a dose of at most about 4 mg/kg. In some embodiments, a checkpoint inhibitor can be administered at a dose of at most about 5 mg/kg.

In some embodiments, a checkpoint inhibitor can be administered at a dose of from about 25 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1,000 mg per dose administered. In some embodiments, a checkpoint inhibitor can be administered at a dose of from about 50 mg to about 250 g per dose administered. In some embodiments, a checkpoint inhibitor can be administered at a dose of from about 250 mg to about 500 mg per dose administered. In some embodiments, a checkpoint inhibitor can be administered at a dose of from about 500 mg to about 750 mg per dose administered.

In some embodiments, a checkpoint inhibitor can be administered at a dose of at least about 25 mg, at least about 50 mg, at least about 100 mg, at least about 150 mg, at least about 200 mg, at least about 250 mg, at least about 300 mg, at least about 350 mg, at least about 400 mg, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, or at least about 1,000 mg. In some embodiments, a checkpoint inhibitor can be administered at a dose of at least about 240 mg. In some embodiments, a checkpoint inhibitor can be administered at a dose of at least about 250 mg. In some embodiments, a checkpoint inhibitor can be administered at a dose of at least about 300 mg. In some embodiments, a checkpoint inhibitor can be administered at a dose of at least about 450 mg. In some embodiments, a checkpoint inhibitor can be administered at a dose of at least about 480 mg. In some embodiments, a checkpoint inhibitor can be administered at a dose of at least about 500 mg.

In some embodiments, a checkpoint inhibitor can be administered at a dose of about 25 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg. In some embodiments, a checkpoint inhibitor can be administered at a dose of about 240 mg. In some embodiments, a checkpoint inhibitor can be administered at a dose of about 250 mg. In some embodiments, a checkpoint inhibitor can be administered at a dose of about 300 mg. In some embodiments, a checkpoint inhibitor can be administered at a dose of about 450 mg. In some embodiments, a checkpoint inhibitor can be administered at a dose of about 480 mg. In some embodiments, a checkpoint inhibitor can be administered at a dose of about 500 mg.

In some embodiments, a checkpoint inhibitor is administered at a concentration of from about 1 mg/mL to about 2 mg/mL, from about 2 mg/mL to about 3 mg/mL, from about 3 mg/mL to about 4 mg/mL, from about 4 mg/mL to about 5 mg/mL, from about 5 mg/mL to about 6 mg/mL, from about 6 mg/mL to about 7 mg/mL, from about 7 mg/mL to about 8 mg/mL, from about 8 mg/mL to about 9 mg/mL, or from about 9 mg/mL to about 10 mg/mL. In some embodiments, a checkpoint inhibitor is administered at a concentration of from about 9 mg/mL to about 10 mg/mL. In some embodiments, a checkpoint inhibitor is administered at a concentration of at least about 1 mg/mL, at least about 1.5 mg/mL, at least about 2 mg/mL, at least about 2.5 mg/mL, at least about 3 mg/mL, at least about 3.5 mg/mL, at least about 4 mg/mL, at least about 4.5 mg/mL, at least about 5 mg/mL, at least about 5.5 mg/mL, at least about 6 mg/mL, at least about 6.5 mg/mL, at least about 7 mg/mL, at least about 7.5 mg/mL, at least about 8 mg/mL, at least about 8.5 mg/mL, at least about 9 mg/mL, at least about 9.5 mg/mL, or at least about 10 mg/mL. In some embodiments, a checkpoint inhibitor is administered at a concentration of about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, or about 10 mg/mL. In some embodiments, a checkpoint inhibitor is administered at a concentration of about 10 mg/mL

In some embodiments, the volume of infusion is at most about 5 mL/kg, at most about 4.5 mL/kg, at most about 4 mL/kg, at most about 3.5 mL/kg, at most about 3 mL/kg, at most about 2.5 mL/kg, at most about 2 mL/kg, at most about 1.5 mL/kg, at most about 1 mL/kg, or at most about 0.5 mL/kg of body weight. In some embodiments, the volume of infusion is at most about 4 mL/kg of body weight. In some embodiments, the volume of infusion is at most about 3 mL/kg of body weight.

In some embodiments, the total volume of infusion is at most about 200 mL, at most about 175 mL, at most about 150 mL, at most about 125 mL, at most about 100 mL, at most about 75 mL, at most about 50 mL, or at most about 25 mL. In some embodiments, the total volume of infusion is at most about 160 mL. In some embodiments, the total volume of infusion is at most about 125 mL. In some embodiments, the total volume of infusion is at most about 100 mL.

In some embodiments, a checkpoint inhibitor can be administered at a dose of at least about 240 mg. In some embodiments, a checkpoint inhibitor can be administered at a dose of at least about 480 mg.

In some embodiments, the checkpoint inhibitor is administered intravenously. In some embodiments, the checkpoint inhibitor is administered orally.

In some embodiments, the checkpoint inhibitor is administered once every day, once every 2 days, once every 3 days, once every 4 days, once every 5 days, once every 6 days, once every week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, or once every 12 weeks. In some embodiments, the checkpoint inhibitor is administered once every week. In some embodiments, the checkpoint inhibitor is administered once every week. In some embodiments, the checkpoint inhibitor is administered once every 2 weeks. In some embodiments, nivolumab is administered once every 4 weeks. In some embodiments, the checkpoint inhibitor is administered once every 6 weeks. In some embodiments, the checkpoint inhibitor is administered once every 8 weeks. In some embodiments, the checkpoint inhibitor is administered once every 12 weeks.

In some embodiments, the checkpoint inhibitor is administered 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, or about 28 times over a 28-day cycle. In some embodiments, the checkpoint inhibitor is administered 1 time over a 28-day cycle. In some embodiments, the checkpoint inhibitor is administered 2 times over a 28-day cycle. In some embodiments, the checkpoint inhibitor is administered 3 times over a 28-day cycle. In some embodiments, the checkpoint inhibitor is administered 4 times over a 28-day cycle. In some embodiments, the checkpoint inhibitor is administered on day 1 of a 28 day cycle. In some embodiments, the checkpoint inhibitor is administered on day 15 of a 28 day cycle. In some embodiments, the checkpoint inhibitor is administered on day 22 of a 28 day cycle. In some embodiments, the checkpoint inhibitor is administered on day 1 and day 15 of a 28 day cycle.

In some embodiments, the checkpoint inhibitor is administered over about 15 minutes, about 30 minutes, about 45 minutes, about 60 minutes, about 90 minutes, or about 120 minutes. In some embodiments, the checkpoint inhibitor is administered over 30 minutes. In some embodiments, the checkpoint inhibitor is administered over 60 minutes. In some embodiments, the checkpoint inhibitor is administered over 90 minutes. In some embodiments, the checkpoint inhibitor is administered over 120 minutes.

In some embodiments, nivolumab is administered over 30 minutes. In some embodiments, nivolumab is administered over 60 minutes. In some embodiments, nivolumab is administered at a dose of about 240 mg every 2 weeks over 30 minutes. In some embodiments, nivolumab is administered at a dose of about 480 mg every 2 weeks over 60 minutes. In some embodiments, nivolumab is administered at a dose of about 480 mg every 4 weeks over 30 minutes for the first 16 weeks, followed by 480 mg every 4 weeks over 30 minutes.

### Diseases

A method disclosed herein can be used to treat, for example, an infectious disease, a proliferative disease, a cancer, a solid tumor, a liquid tumor, non-small cell lung cancer, melanoma, colorectal cancer, head and neck cancer, bladder cancer, or pancreatic cancer. In some embodiments, compounds of the invention can be used to treat cancer in a subject.

Subjects can be, for example, elderly adults, adults, adolescents, pre-adolescents, children, toddlers, infants, neonates, and non-human animals. In some embodiments, a subject is a patient.

A compound of the invention can, for example, slow the proliferation of cancer cell lines, or kill cancer cells. In some embodiments, the cancer comprises 1, 2, 3, 4, 5, 6, 7, 8, 9, or 10 mutations. In some embodiments, the cancer comprises 1 mutation. In some embodiments, the cancer comprises 2 mutations. In some embodiments, the cancer comprises 3 mutations. In some embodiments, the cancer comprises 4 mutations. In some embodiments, the cancer comprises 5 mutations.

In some embodiments, the cancer comprises a KRAS mutation. In some embodiments, the cancer comprises a G12V mutation. In some embodiments, the cancer comprises a G12D mutation. In some embodiments, the cancer comprises a G12C mutation. In some embodiments, the cancer comprises a I46T mutation.

Non-limiting examples of cancer that can be treated by a compound of the invention include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

A tumor response due to a method disclosed herein can be measured based on the RECIST classification of responses.

### EXAMPLES

### Example 1: Study to Evaluate an Oral Pharmaceutical Composition Disclosed Herein for Treating a Disease in a Subject

Approximately 30 patients are enrolled in a dose escalation study of rigosertib in non-small cell lung cancer to investigate doses of rigosertib in combination with nivolumab. The dose of nivolumab is 240 mg every two weeks or 480 mg every four weeks. The next rigosertib dose cohort is 840 mg in the morning and 560 mg in the afternoon, given at least 2 hours after the prior meal and 1 hour before each subsequent meal. Dose increments in the dose escalation 3+3 study are 280 mg each, with the morning dose being raised first, then the afternoon dose being raised in the next cohort, for example, 840 mg/560 mg, then 840 mg/840 mg, then 1,120 mg/840 mg, etc. (morning and afternoon, respectively). Per 3+3 dose escalation studies, the MTD and RP2D are determined based on DLTs and after determination of the RP2D, an expansion cohort of up to 12 patients is enrolled. In addition to the assessment of safety and tolerability, efficacy is measured by the overall response rate as assessed by iRECIST.

Following determination of the RP2D a randomized, controlled phase 2 study is performed with rigosertib combination with nivolumab. Approximately 200 patients are enrolled in a 1:1 ratio to receive either nivolumab+placebo or nivolumab+rigosertib. Primary outcome is the statistical comparison between progression-free survival (PFS) rates, with overall survival (OS) as the secondary endpoint.

### Example 2: Study to Evaluate an Intravenous Pharmaceutical Composition Disclosed Herein for Treating a Disease in a Subject

Approximately 30 patients are enrolled in a dose escalation study of IV rigosertib in colorectal cancer to determine the MTD in combination with nivolumab. The study is performed in combination with standard dose check point inhibition as per the label. The initial dose of IV rigosertib given in combination is 1,200 mg/24 hours given as a continuous infusion (CIV) for days 1-3 of a 14-day cycle. The next rigosertib dose cohort is 1,500 mg/24 hours as a continuous infusion (CIV) for days 1-3 of a 14-day cycle. Dose increments in the dose escalation 3+3 study are 300 mg/24 hours each. (1,800 mg, 2,100 mg, 2,400 mg, etc.). Per 3+3 dose escalation studies, the MTD and RP2D are determined based on DLTs and after determination of the RP2D, an expansion cohort of up to 12 patients is enrolled at the RP2D. In addition to the assessment of safety and tolerability, efficacy is measured by the overall response rate as assessed by iRECIST.

Following determination of the RP2D a randomized, controlled phase 2 study is performed in combination with nivolumab. Approximately 200 patients are enrolled in a 1:1 ratio to receive either nivolumab+placebo or nivolumab+rigosertib. Primary outcome is the statistical comparison between progression-free survival (PFS) rates, with overall survival (OS) as the secondary endpoint.

### Example 3: Study to Evaluate PK/PD Results for a Pharmaceutical Composition Disclosed Herein for Treating a Disease in a Subject

Levels of a biomarker or protein are measured in a cancer cell specimen collected before beginning treatment and at the end of treatment. The effects of rigosertib on the RAS mutation in the tumor and circulating tumor cells are studied, along with the immunological milieu of the patients responding and compared to those who do not respond to the treatment. The specific time points for blood and tissue collection for pharmacodynamic assessments are determined by the protocol. Pharmacodynamic data are tabulated and summarized by individual patient and collectively by dose level. Graphical displays are provided where useful in the interpretation of results.

Results available from previous genetic and biomarker tests, and additional tests of the blood and liquid cancer cell samples for biomarkers relevant to the safety and efficacy of a pharmaceutical composition described herein can be investigated for possible correlation with patient outcome.

### Example 4: Animal Models to Test Activity of Rigosertib in RAS Mutated Cancers with Immune Checkpoint Inhibitors

KRAS has been identified to be mutated in a number of tumor types including lung, GI Tract (colon), pancreas, skin, breast, hematopoietic tissues, gynecological, testis, and urinary tumors. In addition to mutations, other perturbations of KRAS pathway can occur leading to activation of the pathway such as amplification of wild-type KRAS. Identification of specific KRAS show that most mutations primarily occur at the G12 and G13 amino acids with a mutations changing the G to a C (G12C and G13C, respectively). In addition G12D, G12V, G12R and G12A mutations are also clinically relevant and seem to be cancer specific.

Multiple mouse models are employed to test the efficacy of rigosertib in RAS mutated cancers in combination with an immune checkpoint inhibitor. A patient-derived xenograft (PDX) model is used for the experiments, including samples obtained from NSCLC, colo-rectal, and pancreatic cancer patient. The specific PDX model used is determined based on the KRAS mutation status of the patient sample. The tumors are analyzed via genomic sequencing to determine the KRAS mutation and any other driver mutations or amplifications in other known cell cycle or survival signaling pathways.

Patient derived samples are studied in humanized mice. Since optimal activity of checkpoint inhibitors requires an intact immune system, the studies are run in specific immunocompetent mouse models. Humanized mouse models routinely use mice with severe combined immunodeficiency that have been transplanted with hematopoietic stem cells or human peripheral blood cells. After transplantation, the mice are engrafted with the KRAS driven PDX line and treated as described below.

In addition to the humanized models employing PDX models, rigosertib and checkpoint inhibitor combinations are studied in murine syngeneic models. Syngeneic models utilize allografts of KRAS driven tumor cells derived from the exact strain of mouse that are engrafted, which protects the tumors from rejection. A third model uses murine models that are genetically engineered to express human immune components (GEMMS) that also have genetically defined KRAS mutation driven cancers.

The animal assays establish the ability of rigosertib to synergize the activity of the checkpoint inhibitor or act in an additive manner. The studies also address the various dosing and scheduling of rigosertib and how dosing affects the efficacy of the rigosertib-checkpoint inhibitor combination treatments.

The humanized mice are implanted either subcutaneously or orthotopically with patient derived tumor tissue. Syngeneic mice are implanted subcutaneously with KRAS activated cells with a genetic background is identical to the mouse strain being implanted. The tumors are permitted to grow to a tumor volume is greater than or equal to 50 mm³. Once are enough animals harbor tumors with similar tumor volumes, the mice are treated with rigosertib and the checkpoint inhibitor.

Rigosertib is administered at various doses and schedules. Rigosertib is administered by oral gavage at 50 mg/kg (HED=243.9), 100 mg/kg (HED: 487.8 mg), 150 mg/kg (HED 731.7 mg), 200 mg/kg (HED=975 mg), 250 mg/kg (HED=1219.8 mg), or 300 mg/kg (HED=1463.4 mg). The schedule is BID at each dose. The schedule is further modified to mimic the scheduling in humans and reduce possible toxicity, such that the schedules follow: 150 mg/kg, 200 mg/kg, 250 mg/kg, and 300 mg/kg in the morning followed by pairing with a higher dose in the afternoon (TABLE 4).

TABLE 4

| AM Dose (mg/kg) | PM Dose (mg/kg) |
|---|---|
| 100 | 150, 200, 250, 300 |
| 150 | 200, 250, 300 |
| 200 | 250, 300 |
| 250 | 300 |

The animals dosed with checkpoint inhibitors are administered a dose at a schedule that is clinically relevant. For example, nivolumab is tested in a dose range between 5-30 mg/kg administered 1-2 times per week by inftraperitoneal injection. Pembrolizumab is administered 5-10 mg/kg every 3-5 days by intraperitoneal injection. For syngeneic models, mouse anti-PD-1, PD-L1 and CTLA-4 clones are used for optimal antigen specific recognition. In addition to the combination groups, control groups consist of rigosertib alone, the checkpoint inhibitor alone, and an appropriate vehicle control.

Assessment of the growth inhibitory activity of each agent is determined by two criteria: tumor growth and increase in life span (ILS). Tumor growth is monitored by determining a tumor volume. Tumor sizes are calculated according to the formula W²×L/2 (L=length and W=the perpendicular width of the tumor, L>W). The second criteria (used for the leukemia model) measures an increase in ILS. ILS is calculated using the following formula: (mean survival of treated/(mean survival of vehicle control)−1×100.

### EMBODIMENTS

The following non-limiting embodiments provide illustrative examples of the invention, but do not limit the scope of the invention.

Embodiment 1. A method of treating a condition comprising administering to a subject in need thereof:
a) a therapeutically-effective amount of a compound of the formula:

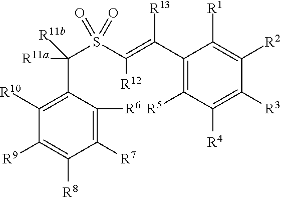

or a pharmaceutically-acceptable salt or zwitterion thereof,
wherein:
each R¹, R², R³, R⁴, R⁵, R⁶, R⁷, R⁸, R⁹, R¹⁰, R^{11a}, R^{11b}, R¹², and R¹³ is independently alkyl, alkenyl, alkynyl, alkoxy, aryl, heteroaryl, heterocyclyl, —C(O)R^{x}, —C(O)OR^{x}, —C(O)NR^{x}R^{y}, —OR^{x}, —SR^{x}, —NR^{x}R^{y}, —NR^{x}C(O)R^{y}, —OC(O)R^{x}, or —SiR^{x}R^{y}R^{z}, each of which is independently substituted or unsubstituted; or hydrogen or halogen; and
each R^{x}, R^{y}, and R^{z} is independently alkyl, alkenyl, alkynyl, aryl, heteroaryl, or heterocyclyl, each of which is independently substituted or unsubstituted; or hydrogen or halogen, and
b) a therapeutically-effective amount of a checkpoint inhibitor.

Embodiment 2. The method of embodiment 1, wherein the condition is a cancer.

Embodiment 3. The method of embodiment 2, wherein the cancer comprises a KRAS mutation.

Embodiment 4. The method of embodiment 3, wherein the KRAS mutation is G12V.

Embodiment 5. The method of embodiment 3, wherein the KRAS mutation is G12D.

Embodiment 6. The method of embodiment 3, wherein the KRAS mutation is G12C.

Embodiment 7. The method of embodiment 3, wherein the KRAS mutation is I46T.

Embodiment 8. The method of embodiment 2, wherein the cancer does not comprise a G12C KRAS mutation.

Embodiment 9. The method of any one of embodiments 1-8, wherein the condition is non-small cell lung carcinoma.

Embodiment 10. The method of any one of embodiments 1-8, wherein the condition is lung adenocarcinoma.

Embodiment 11. The method of any one of embodiments 1-10, wherein the administering the compound is intravenous.

Embodiment 12. The method of any one of embodiments 1-10, wherein the administering the compound is oral.

Embodiment 13. The method of any one of embodiments 1-12, wherein the therapeutically-effective amount of the compound is from about 100 mg to about 3,000 mg.

Embodiment 14. The method of any one of embodiments 1-12, wherein the therapeutically-effective amount of the compound is at least about 560 mg.

Embodiment 15. The method of any one of embodiments 1-12, wherein the therapeutically-effective amount of the compound is at least about 840 mg.

Embodiment 16. The method of any one of embodiments 1-12, wherein the therapeutically-effective amount of the compound is at least about 1,120 mg.

Embodiment 17. The method of any one of embodiments 1-12, wherein the therapeutically-effective amount of the compound is at least about 1,200 mg.

Embodiment 18. The method of any one of embodiments 1-12, wherein the therapeutically-effective amount of the compound is at least about 1,500 mg.

Embodiment 19. The method of any one of embodiments 1-12, wherein the therapeutically-effective amount of the compound is at least about 1,800 mg.

Embodiment 20. The method of any one of embodiments 1-19, wherein the compound is administered once a day.

Embodiment 21. The method of any one of embodiments 1-19, wherein the compound is administered twice a day.

Embodiment 22. The method of any one of embodiments 1-19 or 21, wherein a first therapeutically-effective amount of the compound is administered to the subject in a morning of a day, and a second therapeutically-effective amount of the compound is administered to the subject in an afternoon of the day.

Embodiment 23. The method of any one of embodiments 1-19 or 21, wherein a first therapeutically-effective amount of the compound is administered to the subject in a morning of a day, and a second therapeutically-effective amount of the compound is administered to the subject in an evening of the day.

Embodiment 24. The method of embodiment 22 or 23, wherein the first therapeutically-effective amount and the second therapeutically-effective amount are the same.

Embodiment 25. The method of embodiment 22 or 23, wherein the first therapeutically-effective amount and the second therapeutically-effective amount are different.

Embodiment 26. The method of embodiment 22 or 23, wherein the first therapeutically-effective amount is greater than the second therapeutically-effective amount is.

Embodiment 27. The method of embodiment 22 or 23, wherein the first therapeutically-effective amount is lesser than the second therapeutically-effective amount is.

Embodiment 28. The method of any one of embodiments 1-19, wherein the administering the compound occurs three times a day.

Embodiment 29. The method of any one of embodiments 1-28, wherein the administering the compound occurs at least an hour before the subject consumes food.

Embodiment 30. The method of any one of embodiments 1-28, wherein the administering the compound occurs at least hour after the subject consumes food.

Embodiment 31. The method of any one of embodiments 1-30, wherein the administering the compound occurs at least one hour before the subject consumes a drink.

Embodiment 32. The method of any one of embodiments 1-30, wherein the administering the compound occurs at least one hour after the subject consumes a drink.

Embodiment 33. The method of any one of embodiments 1-32, wherein each R¹, R³, and R⁵ is independently OR^{x}.

Embodiment 34. The method of any one of embodiments 1-33, wherein each R^{x} is independently alkyl, aryl, heteroaryl, or heterocyclyl, each of which is independently substituted or unsubstituted.

Embodiment 35. The method of any one of embodiments 1-33, wherein each R^{x} is independently substituted or unsubstituted C₁₋₆ alkyl.

Embodiment 36. The method of any one of embodiments 1-35, wherein each R^{x} is independently C₁ alkyl substituted with hydroxy, sulfhydryl, halogen, an amino group, a nitro group, cyano, a sulfoxide group, a sulfone group, a sulfonamide group, a carboxyl group, a carboxylic acid, a carboxaldehyde group, alkoxy, aryl, a heterocyclyl group, an acyl group, an amide, or an ester.

Embodiment 37. The method of any one of embodiments 1-36, wherein each R^{x} is independently methyl.

Embodiment 38. The method of any one of embodiments 1-37, wherein each R², R⁴, R⁶, R⁹, and R¹⁰ is independently hydrogen.

Embodiment 39. The method of any one of embodiments 1-38, wherein each R^{11a} and R^{11b} is independently hydrogen.

Embodiment 40. The method of any one of embodiments 1-39, wherein each R¹² and R¹³ is independently hydrogen.

Embodiment 41. The method of any one of embodiments 1-40, wherein the compound has the formula:

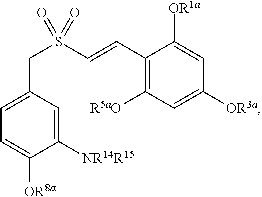

wherein:
each R^{1a}, R^{3a}, R^{5a}, and R^{8a} is independently substituted or unsubstituted C₁₋₈ alkyl; and
each R¹⁴ and R¹⁵ is independently alkyl, alkoxy, aryl, heteroaryl, or heterocyclyl, each of which is independently substituted or unsubstituted; or hydrogen.

Embodiment 42. The method of embodiment 41, wherein each Ria R^{3a}, R^{5a}, and R^{8a} is independently methyl.

Embodiment 43. The method of embodiment 41, wherein R¹⁴ is hydrogen.

Embodiment 44. The method of embodiment 41, wherein R¹⁵ is C₁ alkyl substituted with hydroxy, sulfhydryl, halogen, an amino group, a nitro group, cyano, a sulfoxide group, a sulfone group, a sulfonamide group, a carboxyl group, a carboxylic acid, a carboxaldehyde group, alkoxy, aryl, a heterocyclyl group, an acyl group, an amide, or an ester.

Embodiment 45. The method of embodiment 41, wherein R¹⁵ is CH₂COOH.

Embodiment 46. The method of any one of embodiments 1-45, wherein the compound is ((E)-2-(5-((2,4,6-trimethoxystyryl sulfonyl)methyl)-2-methoxyphenylamino)acetic acid or a pharmaceutically-acceptable salt or zwitterion thereof.

Embodiment 47. The method of any one of embodiments 1-45, wherein the compound is sodium (E)-2-(5-((2,4,6-trimethoxystyrylsulfonyl)methyl)-2-methoxyphenylamino)acetate.

Embodiment 48. The method of any one of embodiments 1-47, wherein the administering the checkpoint inhibitor is intravenous.

Embodiment 49. The method of any one of embodiments 1-47, wherein the administering the checkpoint inhibitor is oral.

Embodiment 50. The method of any one of embodiments 1-49, wherein the checkpoint inhibitor is a cell-surface checkpoint inhibitor.

Embodiment 51. The method of any one of embodiments 1-49, wherein the checkpoint inhibitor is a CTLA-4 inhibitor.

Embodiment 52. The method of any one of embodiments 1-49, wherein the checkpoint inhibitor is a PD-1 inhibitor.

Embodiment 53. The method of any one of embodiments 1-49, wherein the checkpoint inhibitor is a PD-L1 inhibitor.

Embodiment 54. The method of any one of embodiments 1-49, wherein the checkpoint inhibitor is an intracellular checkpoint inhibitor.

Embodiment 55. The method of any one of embodiments 1-49, wherein the checkpoint inhibitor is cytokine-inducible SH2-containing protein (CISH).

Embodiment 56. The method of any one of embodiments 1-49, wherein the checkpoint inhibitor is Nivolumab.

Embodiment 57. The method of any one of embodiments 1-49, wherein the checkpoint inhibitor is pembrolizumab.

Embodiment 58. The method of any one of embodiments 1-49, wherein the checkpoint inhibitor is ipilimumab.

Embodiment 59. The method of any one of embodiments 1-49, wherein the checkpoint inhibitor is atezolizumab.

Embodiment 60. The method of any one of embodiments 1-59, wherein the therapeutically-effective amount of the checkpoint inhibitor is from about 100 mg to about 1,000 mg.

Embodiment 61. The method of any one of embodiments 1-59, wherein the therapeutically-effective amount of the checkpoint inhibitor is about 240 mg.

Embodiment 62. The method of any one of embodiments 1-59, wherein the therapeutically-effective amount of the checkpoint inhibitor is about 480 mg.

Embodiment 63. The method of any one of embodiments 1-59, wherein the therapeutically-effective amount of the checkpoint inhibitor is from about 2 mg/kg to about 12 mg/kg.

Embodiment 64. The method of any one of embodiments 1-59, wherein the therapeutically-effective amount of the checkpoint inhibitor is about 2 mg/kg.

Embodiment 65. The method of any one of embodiments 1-59, wherein the therapeutically-effective amount of the checkpoint inhibitor is about 3 mg/kg.

Embodiment 66. The method of any one of embodiments 1-59, wherein the therapeutically-effective amount of the checkpoint inhibitor is about 10 mg/kg.

Embodiment 67. The method of any one of embodiments 1-66, wherein the checkpoint inhibitor is administered over 30 minutes.

Embodiment 68. The method of any one of embodiments 1-66, wherein the checkpoint inhibitor is administered over 60 minutes.

Embodiment 69. The method of any one of embodiments 1-68, wherein the checkpoint inhibitor is administered once every 2 weeks.

Embodiment 70. The method of any one of embodiments 1-68, wherein the administering of the checkpoint inhibitor occurs once every 4 weeks.

Embodiment 71. The method of any one of embodiments 1-68, wherein the administering of the compound occurs on 21 consecutive days of a 28-day cycle.

Embodiment 72. The method of any one of embodiments 1-68, wherein the administering of the checkpoint inhibitor occurs on day 1 and day 15 of a 28 day cycle.

1-72. (canceled)

73. A method of treating a cancer comprising
a) administering to a subject in need thereof a therapeutically-effective amount of a compound that is ((E)-2-(5-((2,4,6-trimethoxystyryl sulfonyl)methyl)-2-methoxyphenylamino)acetic acid or a pharmaceutically-acceptable salt or zwitterion thereof, wherein the therapeutically-effective amount of the compound is at least about 100 mg; and
b) administering to the subject a therapeutically-effective amount of a checkpoint inhibitor.

74. The method of claim 73, wherein the cancer comprises a KRAS mutation.

75. The method of claim 74, wherein the KRAS mutation is G12V.

76. The method of claim 74, wherein the KRAS mutation is G12D.

77. The method of claim 74, wherein the KRAS mutation is G12C.

78. The method of claim 74, wherein the KRAS mutation is I46T.

79. The method of claim 73, wherein the cancer does not comprise a G12C KRAS mutation.

80. The method of claim 73, wherein the cancer is non-small cell lung carcinoma.

81. The method of claim 73, wherein the cancer is lung adenocarcinoma.

82. The method of claim 73, wherein the therapeutically effective amount of the compound is from about 100 mg to about 3,000 mg.

83. The method of claim 73, wherein the administering the compound is intravenous.

84. The method of claim 73, wherein the administering the compound is oral.

85. The method of claim 73, wherein the compound is sodium (E)-2-(5-((2,4,6-trimethoxystyrylsulfonyl)methyl)-2-methoxyphenylamino)acetate.

86. The method of claim 73, wherein the checkpoint inhibitor is a cell-surface checkpoint inhibitor.

87. The method of claim 73, wherein the checkpoint inhibitor is a CTLA-4 inhibitor.

88. The method of claim 73, wherein the checkpoint inhibitor is a PD-1 inhibitor.

89. The method of claim 73, wherein the checkpoint inhibitor is a PD-L1 inhibitor.

90. The method of claim 73, wherein the checkpoint inhibitor is an intracellular checkpoint inhibitor.

91. The method of claim 73, wherein the checkpoint inhibitor is cytokine-inducible SH2-containing protein (CISH).

92. The method of claim 73, wherein the checkpoint inhibitor is Nivolumab.

93. The method of claim 73, wherein the checkpoint inhibitor is pembrolizumab.

94. The method of claim 73, wherein the checkpoint inhibitor is ipilimumab.

95. The method of claim 73, wherein the checkpoint inhibitor is atezolizumab.

96. The method of claim 73, wherein the administering of the compound occurs on 21 consecutive days of a 28-day cycle.

97. The method of claim 73, wherein the administering of the checkpoint inhibitor occurs on day 1 and day 15 of a 28 day cycle.
